(19) United States
(12) Patent Application Publication
(10) Pub. No.: US 2025/0299782 A1
(43) Pub. Date: Sep. 25, 2025

(54) CROSSLINKING REACTION SIMULATION DEVICE AND INTERNAL BUBBLE ESTIMATION DEVICE

(71) Applicant: Sumitomo Riko Company Limited, Komaki-shi (JP)

(72) Inventors: Hiroshi Sumida, Aichi (JP); Shota Nishimura, Aichi (JP); Kiyoshi Sakai, Aichi (JP); Maho Noguchi, Aichi (JP); Takanori Sugiura, Aichi (JP); Tetsuya Watanabe, Aichi (JP); Norihito Kimura, Aichi (JP)

(73) Assignee: Sumitomo Riko Company Limited, Komaki-shi (JP)

(21) Appl. No.: 19/226,210

(22) Filed: Jun. 3, 2025

Related U.S. Application Data
(63) Continuation of application No. PCT/JP2024/006995, filed on Feb. 27, 2024.

(30) Foreign Application Priority Data
Mar. 29, 2023 (JP) 2023-052538
Mar. 29, 2023 (JP) 2023-052539
Mar. 29, 2023 (JP) 2023-052540

(51) Int. Cl. G16C 20/10; G06F 17/17; G16C 20/80

(52) U.S. Cl. CPC G16C 20/10; G06F 17/17; G16C 20/80

(57) ABSTRACT

A crosslinking reaction simulation device including: a heat transfer analysis unit configured to perform a heat transfer analysis during a crosslinking reaction of a polymer portion of a target work model; and a crosslinking reaction analysis unit configured to perform analysis of a reaction rate of a crosslinking reaction of the polymer portion, and the crosslinking reaction analysis unit includes a temperature acquisition unit that acquires, as a result of the heat transfer analysis, a temperature at each time for each element of the polymer portion of the target work model in the crosslinking reaction, and, a reaction rate calculation processing unit configured to calculate an equivalent reaction amount of the polymer portion at each time, and calculates the reaction rate of the crosslinking reaction of the polymer portion based on the calculated equivalent reaction amount.

### CROSS-REFERENCE

The application is Continuation of International Application No. PCT/JP2024/006995 filed on Feb. 27, 2024, which claims priority to Japanese Patent Application No. 2023-052538 filed on Mar. 29, 2023, Japanese Patent Application No. 2023-052539 filed on Mar. 29, 2023, and Japanese Patent Application No. 2023-052540 filed on Mar. 29, 2023. The entire contents of which are incorporated herein by reference.

### BACKGROUND

### (1) Technical Field

The present disclosure relates to a crosslinking reaction simulation device and an internal bubble estimation device.

### (2) Description of Related Art

Conventionally, improvement of physical properties of chemical substances has been performed by causing a polymer to undergo a crosslinking reaction. Examples of such a polymer include rubber. In the rubber, a three-dimensional network crosslinked structure is formed between rubber molecular chains or in the molecular chains by adding sulfur, another crosslinking agent, a vulcanization promoter, and the like to raw material rubber, and performing heating.

Techniques for estimating the vulcanization degree of rubber, that is, the reaction rate of a crosslinking reaction of rubber include a technique described in Non Patent Literature 1. The above technique estimates the vulcanization degree from the temperature history of rubber based on the Arrhenius equation, and estimates appropriate vulcanization conditions (mold temperature, vulcanization time).

### PRIOR ART LITERATURE

### Non Patent Literature

Non Patent Literature 1: Toshio Arimatsu, “Practice of Vulcanization Process Design”, Journal of the Society of Rubber Science and Technology, Japan, Vol. 59, No. 3, (1986)

### SUMMARY

However, since the above technique is not sufficiently high in accurate, the reaction rate of the crosslinking reaction of the rubber cannot be accurately estimated.

When the above technique is used, it is preferable to estimate the temperature history of the polymer during the crosslinking reaction as accurately as possible. However, it has been difficult to estimate the temperature history of the polymer during the crosslinking reaction. This will be described below.

For example, when a crosslinking reaction is caused in a state where a polymer is arranged in a mold, a local temperature of the mold can be managed and measured. However, the thermal conduction from the mold to the polymer and the thermal conduction inside the polymer are affected by the shape of the polymer, the composition of a filler contained in the polymer, and the like. Therefore, when the shape of the product is different, the temperature history of the rubber is different for each product. Furthermore, when the composition of the filler is different even in a product having the same shape, the temperature history of the rubber is different for each product having a different composition. In particular, when the thermal diffusivity of the polymer and the thermal diffusivity of the filler are different, it is very difficult to estimate the temperature history of the polymer.

A rubber product is made by molding and vulcanization, and a gas originally dissolved in rubber or a gas generated by a vulcanization reaction is dissolved in rubber under a high temperature and high pressure condition of vulcanization. When the mold is opened, the pressure applied to the rubber is reduced, and the solubility of the gas in the rubber is reduced, and therefore, a bubble is generated in the rubber in a state where vulcanization is not sufficiently progressed. When the vulcanization time is lengthened and the vulcanization progresses, a bubble is not generated.

In vulcanization of an actual product, the total amount of heat received is different due to a difference in the temperature rise history in a point inside of the product having a different distance from a mold that is a heat source, and the vulcanization progress status is different even in the same vulcanization time. Therefore, in the slowest vulcanization part, it is necessary to perform vulcanization until the time when generation of bubbles is not observed. The vulcanization degree of the rubber in the slowest vulcanization part at the time point when the mold is opened when molding is performed in the vulcanization time until the generation of a bubble is not observed in this slowest vulcanization part is called a blow point vulcanization degree (hereinafter, blow point). In order to determine the vulcanization time of a product, it is important to estimate this blow point.

However, the blow point cannot be estimated by the above technique. For this reason, some conventional techniques have a problem of not being able to accurately predict the vulcanization time of a product. This problem is not limited to rubber, and can be a problem in a crosslinking reaction of a polymer.

The present disclosure has been made in view of such a background, and an object is to provide a crosslinking reaction simulation device and an internal bubble estimation device that can solve any of the above problems.

One aspect of the present disclosure is
a crosslinking reaction simulation device comprising:
a storage unit configured to store data to be used for simulation;
a heat transfer analysis unit configured to perform heat transfer analysis of a polymer portion of a target work model during a crosslinking reaction thereof; and
a crosslinking reaction analysis unit configured to perform analysis of a reaction rate of a crosslinking reaction on the polymer portion using a result of the heat transfer analysis,
wherein the storage unit stores
the target work model including the polymer portion configured to contain a raw material polymer,
an equivalent reaction amount calculation model having a definition of an equivalent reaction amount, as a ratio of a reaction amount of a crosslinking reaction at a target reaction time at a target reaction temperature to a reaction amount of a crosslinking reaction at a reference reaction time at a reference reaction temperature, and including a slope coefficient representing a slope of an Arrhenius plot, and
the slope coefficient set corresponding to a degree of progress of a crosslinking reaction in the polymer portion, and
the crosslinking reaction analysis unit includes
a temperature acquisition unit that acquires, as a result of the heat transfer analysis, a temperature at each time for each element of the polymer portion of the target work model in a crosslinking reaction, and
a reaction rate calculation processing unit configured to calculate, on the basis of the acquired temperature of each element of the polymer portion at each time during the crosslinking reaction, the equivalent reaction amount calculation model, and the slope coefficient corresponding to the degree of progress of the crosslinking reaction at a target time, the equivalent reaction amount of the polymer portion at each time, and to calculate, on the basis of the calculated equivalent reaction amount, a reaction rate of the crosslinking reaction of the polymer portion.

Another aspect of the present disclosure is
a crosslinking reaction simulation device comprising:
a storage unit configured to store data for use in simulation; and
a heat transfer analysis unit configured to perform heat transfer analysis of a polymer portion of a target work model during a crosslinking reaction thereof,
wherein the storage unit stores
a forming mold model,
a target work model including the polymer portion configured to contain a raw material polymer and carbon black, and
a thermal diffusivity characteristic representing a relation between a mass ratio of the carbon black to the raw material polymer and a thermal diffusivity of the polymer portion, and
the heat transfer analysis unit includes
a condition input unit configured to input a mass ratio of the carbon black to the raw material polymer in the target work model and a temperature condition of the forming mold model,
a polymer thermal diffusivity determination unit configured to determine a polymer thermal diffusivity, which is a thermal diffusivity of the polymer portion of the target work model, based on the mass ratio input by the condition input unit and the thermal diffusivity characteristic stored in the storage unit, and
an analysis unit configured to perform heat transfer analysis using the polymer thermal diffusivity determined by the polymer thermal diffusivity determination unit and the temperature condition stored in the storage unit, in a state where the target work model is placed in the forming mold model.

Still another aspect of the present disclosure is
an internal bubble estimation device that is applied to a crosslinking reaction process in which a polymer portion of a target work model is subjected to a crosslinking reaction inside a forming mold model and thereafter is demolded from the forming mold model, and is configured to estimate whether a bubble is generated inside the polymer portion of the target work model in association with demolding from the forming mold model, the internal bubble estimation device comprising:
a storage unit configured to store a crosslinking reaction curve defining a relation between an elapsed time from start of the crosslinking reaction and torque that has a value corresponding to a degree of progress of the crosslinking reaction in the polymer portion of the target work model and is measurable by a crosslinking reaction characteristic tester using a test target polymer material corresponding to the polymer portion;
a reaction rate acquisition unit configured to acquire a reaction rate of a crosslinking reaction of the polymer portion of the target work model;
a torque calculation unit configured to calculate the torque corresponding to the reaction rate acquired by the reaction rate acquisition unit, based on the acquired reaction rate and the crosslinking reaction curve stored in the storage unit; and
an estimation unit configured to estimate, on the basis of the torque during demolding, generation of the bubble inside the polymer portion of the target work model.

According to one aspect of the present disclosure, an equivalent reaction amount of a polymer portion at each time is calculated based on a slope coefficient corresponding to a degree of progress of a crosslinking reaction at a target time, and a reaction rate of the crosslinking reaction of the polymer portion is calculated based on the calculated equivalent reaction amount. This enables the reaction rate of the crosslinking reaction to be accurately estimated as compared with a case where the reaction rate of the crosslinking reaction of the polymer portion is calculated based on the equivalent reaction amount calculated without considering the degree of progress of the crosslinking reaction.

According to another aspect of the present disclosure, since carbon black easily transfers heat as compared with the polymer portion, the mass ratio of carbon black has a large influence on the thermal diffusivity of the polymer portion. According to one aspect of the present disclosure, the polymer thermal diffusivity, which is the thermal diffusivity of the polymer portion of a target work model, can be determined based on the mass ratio of carbon black to the raw material polymer. As a result, the temperature history of the polymer during the crosslinking reaction can be accurately estimated.

According to still another aspect of the present disclosure, generation of a bubble inside the polymer portion of the target work model is estimated based on the torque measurable by the crosslinking reaction characteristic tester. When the pressure at which the polymer portion suppresses the bubble is greater than the pressure at which the bubble expands, the generation of the bubble is suppressed. Therefore, the blow point can be accurately predicted by comparing the torque of the polymer portion with the pressure at which the bubble of the polymer portion is about to expand. This can accurately estimate the vulcanization time of the product.

Note that the reference signs in parentheses in the claims indicate a correspondence relation with specific means described in the embodiments described later, and do not limit the technical scope of the present disclosure.

### BRIEF DESCRIPTION OF DRAWINGS

FIG. 1 is a block diagram illustrating a crosslinking reaction simulation device of a first embodiment.

FIG. 2 is a view illustrating a state where a target work model is arranged in a forming mold model in the first embodiment.

FIG. 3 is a view illustrating the target work model of the first embodiment.

FIG. 4 is a flowchart showing an operation of the crosslinking reaction simulation device of the first embodiment.

FIG. 5 is a view illustrating a temporal change in temperature of each portion in the state where the target work model is arranged in the forming mold model in the first embodiment.

FIG. 6 is a graph showing an actual measurement value and a prediction value of temperature of a polymer portion in a conventional technique.

FIG. 7 is a graph showing an actual measurement value and a prediction value of temperature of a polymer portion in the first embodiment.

FIG. 8 is a graph showing the change amount in thermal conductivity with respect to the content of carbon black in the first embodiment.

FIG. 9 is a view for describing an analysis mesh in a state where the target work model is arranged in the forming mold model in the first embodiment.

FIG. 10 is a schematic view illustrating a structure of a vulcanization tester of the first embodiment.

FIG. 11 is diagrams for describing a method of converting torque measured by the vulcanization tester of the first embodiment into a reaction rate, where (a) is a graph showing a change in torque with respect to the reaction time, and (b) is a graph showing a change in reaction rate with respect to the reaction time.

FIG. 12 is a view for describing a calculation method of a slope coefficient in crosslinking reaction analysis processing of the first embodiment.

FIG. 13 is a view for describing a calculation method of an equivalent reaction amount in the crosslinking reaction analysis processing of the first embodiment.

FIG. 14 is a view for describing a method of calculating the equivalent reaction amount from an equivalent reaction amount increase amount in the crosslinking reaction analysis processing of the first embodiment.

FIG. 15 is a view for illustrating a method of converting the equivalent reaction amount into a reaction rate in the crosslinking reaction analysis processing of the first embodiment.

FIG. 16 is a view illustrating an actual measurement value and a prediction value of a reaction rate of the polymer portion in the conventional technique.

FIG. 17 is a view for describing a method of performing division according to the degree of reaction progress of the crosslinking reaction and calculating activation energy for each division in the first embodiment.

FIG. 18 is a view illustrating an actual measurement value and a prediction value of a reaction rate of the polymer portion in the first embodiment.

FIG. 19 is a view for describing a method of expressing a change amount of the reaction rate with respect to the reaction time in the first embodiment.

FIG. 20 is a view illustrating a change amount of the reaction rate with respect to the reaction time in the first embodiment.

FIG. 21 is a view illustrating a second function of the first embodiment.

FIG. 22 is a view for describing the analysis mesh of the target work model in the first embodiment.

FIG. 23 is a view illustrating a state where an internal bubble is generated in a polymer portion.

FIG. 24 is a view illustrating the reaction rate at a blow point, the reaction rate during demolding, and a final reaction rate in the crosslinking reaction.

FIG. 25 are (a) is a graph showing reaction rates at blow points of a plurality of samples. (b) is a graph showing torque measured by the vulcanization tester at the blow points of the plurality of samples.

FIG. 26 is a view for describing a mechanism of suppressing an internal bubble.

FIG. 27 is a heat map diagram illustrating a reaction rate distribution of the polymer portion in the first embodiment.

FIG. 28 is a view illustrating a change amount of the elastic modulus in a direction perpendicular to an axis of the target work model with respect to the reaction time when the mold temperature is a low temperature in the first embodiment.

FIG. 29 is a view illustrating a change amount of the elastic modulus in the direction perpendicular to the axis of the target work model with respect to the reaction time when the mold temperature is a middle temperature in the first embodiment.

FIG. 30 is a view illustrating a change amount of the elastic modulus in the direction perpendicular to the axis of the target work model with respect to the reaction time when the mold temperature is a high temperature in the first embodiment.

FIG. 31 is a view illustrating changes in the elastic modulus of the polymer portion and the generation status of an internal bubble with respect to the reaction time in the first embodiment.

FIG. 32 is a view for visually displaying a generation expectation result of an internal bubble in the first embodiment.

### DETAILED DESCRIPTION OF PREFERRED EMBODIMENTS

### First Embodiment

### 1. Configuration of Crosslinking Reaction Simulation Device 1

### 1-1. Overall Configuration of Crosslinking Reaction Simulation Device 1

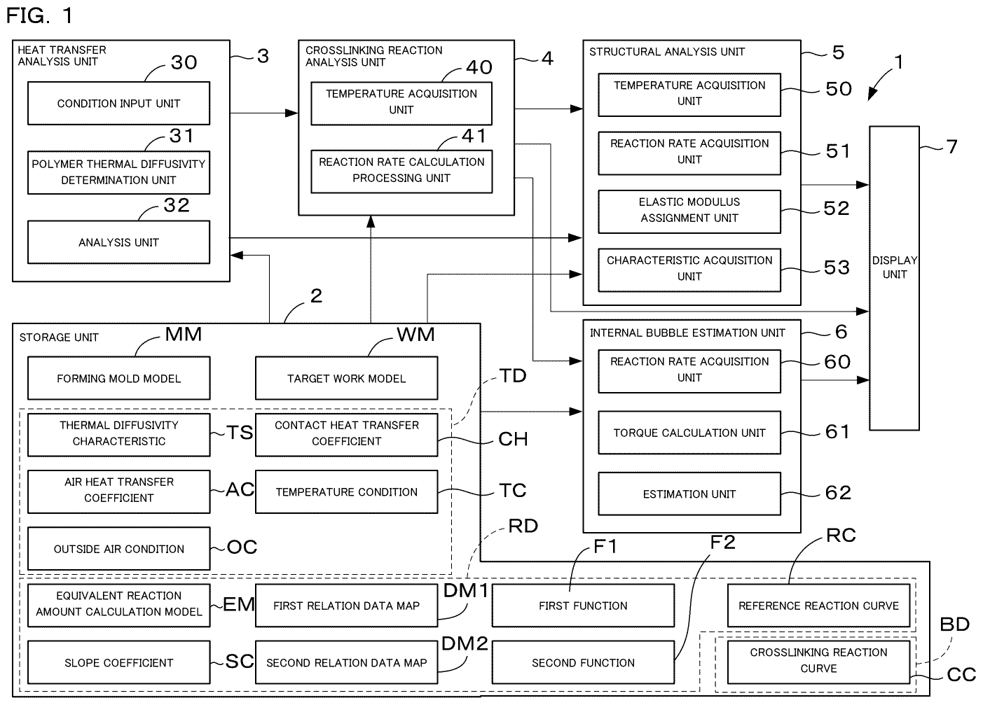

An overall configuration of a crosslinking reaction simulation device 1 of the first embodiment will be described with reference to FIG. 1. The crosslinking reaction simulation device 1 of the present embodiment performs simulation of a crosslinking reaction for crosslinking molecular chains of a raw material polymer.

The raw material polymer is not particularly limited as long as the molecular chains can have a crosslinking reaction with each other, and any polymer can be appropriately selected from thermosetting resins such as phenol resin, epoxy resin, melamine resin, urea resin, unsaturated polyester resin, alkyd resin, silicone resin, and polyurethane, thermoplastic resins such as crosslinked polyethylene and crosslinked polypropylene, synthetic rubbers such as natural rubber, styrene-butadiene rubber, butadiene rubber, and isoprene rubber, and elastomer. In the present embodiment, rubber containing natural rubber and synthetic rubber is used as the polymer. Note that in the case of rubber, when heated in a state where a crosslinking agent such as sulfur is added, molecular chains constituting the rubber are crosslinked, and what is called a vulcanization reaction occurs.

As illustrated in FIG. 1, the crosslinking reaction simulation device 1 includes a storage unit 2, a heat transfer analysis unit 3, a crosslinking reaction analysis unit 4, a structural analysis unit 5, an internal bubble estimation unit 6 (an example of an internal bubble estimation device), and a display unit 7.

### 1-2. Configuration of Storage Unit 2

The storage unit 2 stores data used for simulation. The data includes a forming mold model MM, a target work model WM, heat transfer analysis data TD used by the heat transfer analysis unit 3, crosslinking reaction analysis data RD used by the crosslinking reaction analysis unit 4, and internal bubble estimation data BD used by the internal bubble estimation unit 6.

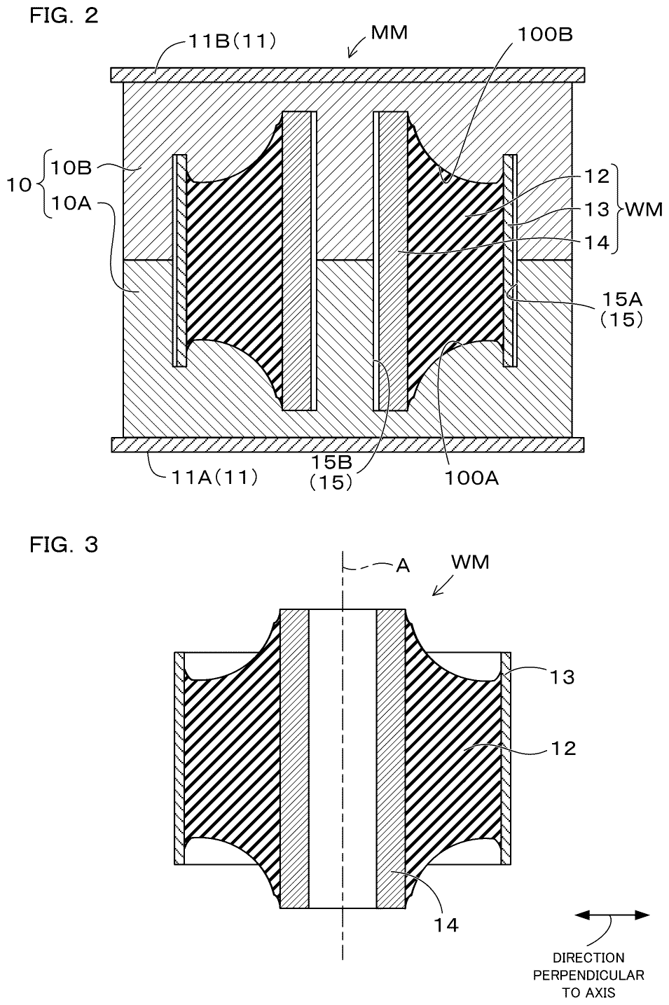

The forming mold model MM will be described with reference to FIG. 2. As illustrated in FIG. 2, the forming mold model MM is configured to include a mold 10 and a heat plate 11 attached to the mold 10. The mold 10 includes a lower mold 10A positioned on the lower side and an upper mold 10B assembled to the lower mold 10A from above. The heat plate 11 is configured to include a lower heat plate 11A attached to a lower surface of the lower mold 10A and an upper heat plate 11B attached to an upper surface of the upper mold 10B. A lower cavity 100A opened upward is formed in the lower mold 10A. An upper cavity 100B opened downward is formed in the upper mold 10B. In a state where the lower mold 10A and the upper mold 10B are assembled, the target work model WM is arranged in a space formed by the lower cavity 100A and the upper cavity 100B.

The target work model WM will be described with reference to FIG. 3. As illustrated in FIG. 3, the target work model WM includes a polymer portion 12. The polymer portion 12 contains a raw material polymer. Furthermore, the polymer portion 12 may contain an additive such as an antioxidant, carbon black, or the like. The polymer portion 12 of the present embodiment is configured to contain a raw material polymer and carbon black. The raw material polymer is not particularly limited, and any material such as rubber or thermosetting resin can be appropriately selected. In the present embodiment, the raw material polymer is made of rubber. The polymer portion 12 of the present embodiment is a rubber portion configured to exhibit anti-vibration performance, and the target work model WM is a model of an anti-vibration rubber device. However, the polymer portion 12 may be configured to contain a thermosetting resin as a raw material polymer, or may be configured not to contain carbon black.

The polymer portion 12 of the present embodiment is formed in a tubular shape along an axis A extending in an up-down direction. An outer joint member 13 (an example of a joint member) formed in a cylindrical shape extending in the up-down direction is arranged on the outer peripheral portion of the polymer portion 12. The outer joint member 13 is made of metal, resin, or as a composite of metal and resin. The outer joint member 13 of the present embodiment is made of metal, and is joined to the outer peripheral portion of the polymer portion 12. An inner joint member 14 (an example of a joint member) formed in a cylindrical shape extending in the up-down direction is arranged on the inner peripheral portion of the polymer portion 12. The inner joint member 14 is made of metal, resin, or as a composite of metal and resin. The inner joint member 14 of the present embodiment is made of metal, and is joined to the inner peripheral portion of the polymer portion 12. The length dimension in the up-down direction of the inner joint member 14 is formed to be larger than the length dimension in the up-down direction of the outer joint member 13. The upper surface and the lower surface of the polymer portion 12 are formed in a concave shape. However, the shape of the polymer portion 12 is not limited to the above shape. The target work model WM may be configured not to include both or one of the outer joint member 13 and the inner joint member 14.

Returning to FIG. 2, in a state where the target work model WM is arranged in the forming mold model MM, a gap 15 is formed between the forming mold model MM and each of the outer joint member 13 and the inner joint member 14.

Returning to FIG. 1, the heat transfer analysis data TD includes a thermal diffusivity characteristic TS indicating a relation between the mass ratio of carbon black to the raw material polymer and the thermal diffusivity of the polymer portion 12, a contact heat transfer coefficient CH, which is a heat transfer coefficient between the forming mold model MM and the outer joint member 13 and the inner joint member 14 in a state where the target work model WM is arranged in the forming mold model MM, an air heat transfer coefficient AC, which is a heat transfer coefficient of air around the target work model WM in a state where the target work model WM is demolded from the forming mold model MM, a temperature condition TC of the forming mold model MM input from a condition input unit 30 described later, and an outside air condition OC including an air temperature around the target work model WM that is demolded.

The crosslinking reaction analysis data RD includes an equivalent reaction amount calculation model EM defined by defining, as an equivalent reaction amount, a ratio of a reaction amount of a crosslinking reaction at a target reaction time at a target reaction temperature to a reaction amount of a crosslinking reaction at a reference reaction time at a reference reaction temperature, and including a slope coefficient representing a slope of an Arrhenius plot, and the slope coefficient SC set corresponding to a degree of progress of a crosslinking reaction of the polymer portion 12. The crosslinking reaction analysis data RD further includes a first relation data map DM1, a second relation data map DM2, a first function F1, a second function F2, and a reference reaction curve RC, which will be described later.

The internal bubble estimation data BD includes a crosslinking reaction curve CC defining a relation between an elapsed time from start of a crosslinking reaction and torque that has a value corresponding to a degree of progress of a crosslinking reaction in the polymer portion 12 of the target work model WM, the torque being measurable by a crosslinking reaction characteristic tester using a test target polymer material corresponding to the polymer portion 12.

### 1-3. Configuration of Heat Transfer Analysis Unit 3

The heat transfer analysis unit 3 configures to perform heat transfer analysis of the polymer portion 12 of the target work model WM during the crosslinking reaction. As illustrated in FIG. 1, the heat transfer analysis unit 3 includes the condition input unit 30, a polymer thermal diffusivity determination unit 31, and an analysis unit 32.

The condition input unit 30 configures to input the mass ratio of carbon black to the raw material polymer in the target work model WM and the temperature condition TC of the forming mold model MM. The condition input unit 30 may be an input device such as a keyboard, a mouse, a trackball, or a joystick, or may be an external storage medium such as a semiconductor memory or a hard disk memory.

The polymer thermal diffusivity determination unit 31 configures to determine the polymer thermal diffusivity, which is the thermal diffusivity of the polymer portion 12 of the target work model WM, based on the mass ratio of carbon black input by the condition input unit 30 and the thermal diffusivity characteristic TS stored in the storage unit 2.

In a state where the target work model WM is arranged in the forming mold model MM, the analysis unit 32 configures to perform heat transfer analysis on the target work model WM in a state of being arranged in the forming mold model MM, using the polymer thermal diffusivity determined by the polymer thermal diffusivity determination unit 31 and the temperature condition TC stored in the storage unit 2.

### 1-4. Configuration of Crosslinking Reaction Analysis Unit 4

The crosslinking reaction analysis unit 4 configures to perform analyses of the reaction rate of the crosslinking reaction on the polymer portion 12 using the result of the heat transfer analysis by the heat transfer analysis unit 3. As illustrated in FIG. 1, the crosslinking reaction analysis unit 4 includes a temperature acquisition unit 40 and a reaction rate calculation processing unit 41.

As a result of the heat transfer analysis by the heat transfer analysis unit 3, the temperature acquisition unit 40 acquires the temperature at each time for each element of the polymer portion 12 of the target work model WM in the crosslinking reaction.

The reaction rate calculation processing unit 41 configured to calculate, on the basis of the acquired temperature of each element of the polymer portion 12 at each time during the crosslinking reaction, the equivalent reaction amount calculation model EM, and the slope coefficient SC corresponding to the degree of progress of the crosslinking reaction at a target time, the equivalent reaction amount of the polymer portion 12 at each time, and to calculate, on the basis of the calculated equivalent reaction amount, a reaction rate of the crosslinking reaction of the polymer portion 12.

### 1-5. Configuration of Structural Analysis Unit 5

The structural analysis unit 5 configures to perform structural analysis using the reaction rate of the crosslinking reaction of the polymer portion 12 analyzed by the crosslinking reaction analysis unit 4. As illustrated in FIG. 1, the structural analysis unit 5 includes a temperature acquisition unit 50, a reaction rate acquisition unit 51, an elastic modulus assignment unit 52, and a characteristic acquisition unit 53. However, the temperature acquisition unit 50 may be omitted.

As a result of the heat transfer analysis by the heat transfer analysis unit 3, the temperature acquisition unit 50 configures to acquire the temperature at each time for each element of the polymer portion 12 of the target work model WM in the crosslinking reaction.

The reaction rate acquisition unit 51 configures to acquire the reaction rate in each element of the polymer portion 12 calculated by the reaction rate calculation processing unit 41 of the crosslinking reaction analysis unit 4.

The elastic modulus assignment unit 52 configures to assign an elastic modulus corresponding to the acquired reaction rate in the polymer portion 12.

The characteristic acquisition unit 53 configures to acquire the characteristic of the target work model WM by performing structural analysis in a state where the elastic modulus is assigned to the polymer portion 12.

### 1-6. Configuration of Internal Bubble Estimation Unit 6

The internal bubble estimation unit 6 configures to be applied to a crosslinking reaction process of causing the polymer portion 12 of the target work model WM to undergo a crosslinking reaction in the forming mold model MM and then demolding the forming mold model MM, and to estimate generation of an internal bubble in association with demolding of the forming mold model MM inside the polymer portion 12 of the target work model WM. As illustrated in FIG. 1, the internal bubble estimation unit 6 includes a reaction rate acquisition unit 60, a torque calculation unit 61, and an estimation unit 62.

The reaction rate acquisition unit 60 configures to acquire the reaction rate of the crosslinking reaction of the polymer portion 12 of the target work model WM. The above reaction rate is a reaction rate at a time point when the mold 10 is opened, that is, a time point when the mold clamping pressure is released. Note that when the polymer portion 12 is rubber, the above reaction rate is also called the vulcanization degree during demolding.

The torque calculation unit 61 configures to calculate torque (also called torque during demolding) corresponding to the reaction rate (reaction rate during demolding) acquired by the reaction rate acquisition unit 60, based on the acquired reaction rate and the crosslinking reaction curve CC stored in the storage unit 2.

The estimation unit 62 configures to compare the torque (torque during demolding) calculated by the torque calculation unit 61 with blow point torque (described in detail later) calculated from a blow point vulcanization degree (described in detail later) in the polymer portion 12, and to estimate whether or not a bubble is generated inside the polymer portion 12.

### 1-7. Configuration of Display Unit 7

The display unit 7 configures to display a value obtained from the reaction rate corresponding to the reaction time based on the analysis result of the crosslinking reaction analysis unit 4. Based on the result of the structural analysis of the structural analysis unit 5, the display unit 7 configures to display characteristics corresponding to the temperature of the forming mold model MM for use in the crosslinking reaction of the polymer portion 12 and an in-mold reaction time from the start of the crosslinking reaction to the demolding of the forming mold model MM.

Based on the estimation result of the internal bubble estimation unit 6, the display unit 7 configures to display the presence or absence of generation of an internal bubble corresponding to the temperature of the forming mold model MM and the in-mold reaction time from the start of the crosslinking reaction to the demolding of the forming mold model MM. Based on the analysis result of the crosslinking reaction analysis unit 4, the display unit 7 displays, in accordance with the presence or absence of generation of the internal bubble, a value obtained from the reaction rate corresponding to the temperature of the forming mold model MM and the in-mold reaction time from the start of the crosslinking reaction to the demolding of the forming mold model MM. Based on the result of the structural analysis of the structural analysis unit 5, the display unit 7 displays, in accordance with the presence or absence of generation of the internal bubble, a characteristic corresponding to the temperature of the forming mold model MM and the in-mold reaction time from the start of the crosslinking reaction to the demolding of the forming mold model MM.

### 2. Overall Operation of Crosslinking Reaction Simulation Device 1

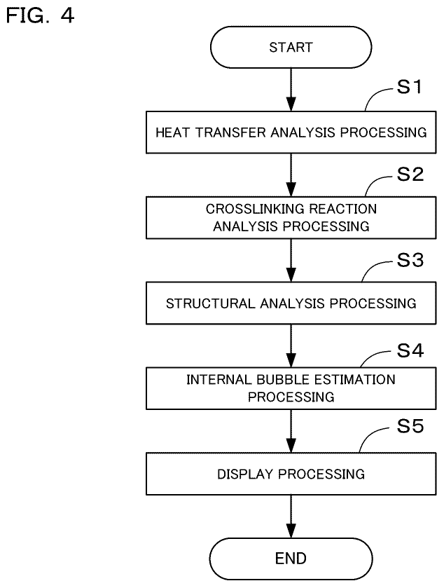

An overall operation of the crosslinking reaction simulation device 1 according to the present embodiment will be described with reference to FIG. 4. However, the following description is an example of the operation of the crosslinking reaction simulation device 1, and the operation of the crosslinking reaction simulation device 1 is not limited to the following description.

When the crosslinking reaction simulation device 1 is activated, heat transfer analysis processing S1 is executed. By the heat transfer analysis processing S1, the temperature at each time is obtained for each element of the polymer portion 12 of the target work model WM in the crosslinking reaction. Details will be described later.

Next, crosslinking reaction analysis processing S2 is performed. In the crosslinking reaction analysis processing S2, the equivalent reaction amount of the polymer portion 12 at each time is calculated based on the temperature at each time for each element of the polymer portion 12 of the target work model WM in the crosslinking reaction, the equivalent reaction amount calculation model EM, and the slope coefficient SC corresponding to the degree of progress of the crosslinking reaction at the target time, which are obtained as a result of the heat transfer analysis, and the reaction rate of the crosslinking reaction of the polymer portion 12 is calculated based on the equivalent reaction amount that is calculated. Details will be described later.

Next, structural analysis processing S3 is executed. In the structural analysis processing S3, the elastic modulus is assigned in accordance with the reaction rate in each element of the polymer portion 12 calculated by the crosslinking reaction analysis unit 4, and the structural analysis is performed in a state where the elastic modulus is assigned to the polymer portion 12, thereby acquiring the characteristic of the target work model WM. Details will be described later.

Next, internal bubble estimation processing S4 is executed. In the internal bubble estimation processing S4, the torque corresponding to the reaction rate of the polymer portion 12 is calculated based on the reaction rate of the polymer portion 12 calculated by the crosslinking reaction analysis processing and the crosslinking reaction curve CC stored in the storage unit 2, and the generation of an internal bubble inside the polymer portion 12 of the target work model WM is estimated based on the torque during demolding. Details will be described later.

Next, display processing S5 is executed. In the display processing S5, the presence or absence of the generation of the internal bubble is displayed in accordance with the temperature of the forming mold model MM and the in-mold reaction time from the start of the crosslinking reaction to the demolding of the forming mold model MM based on the estimation result by the internal bubble estimation unit 6. Based on the analysis result by the crosslinking reaction analysis unit 4, a value obtained from the reaction rate corresponding to the temperature of the forming mold model MM and the in-mold reaction time from the start of the crosslinking reaction to the demolding of the forming mold model MM is displayed in accordance with the presence or absence of generation of the internal bubble. Based on the result of the structural analysis by the structural analysis unit 5, a characteristic corresponding to the temperature of the forming mold model MM and the in-mold reaction time from the start of the crosslinking reaction to the demolding of the forming mold model MM is displayed in accordance with the presence or absence of generation of the internal bubble.

When the display processing S5 ends, the operation of the crosslinking reaction simulation device 1 ends.

### 3. Heat Transfer Analysis Processing S1

### 3-1. Mass Ratio of Carbon Black

Next, details of the heat transfer analysis processing S1 will be described with reference to FIGS. 2 and 5 to 8. As illustrated in FIG. 2, the outer joint member 13 and the inner joint member 14 are arranged in a space formed by the lower cavity 100A and the upper cavity 100B in a state where the lower mold 10A and the upper mold 10B are combined. Furthermore, the raw material polymer is injected into the space formed by the lower cavity 100A and the upper cavity 100B. Thereafter, in the mold 10, a crosslinking reaction of molecular chains constituting the polymer portion 12 progresses.

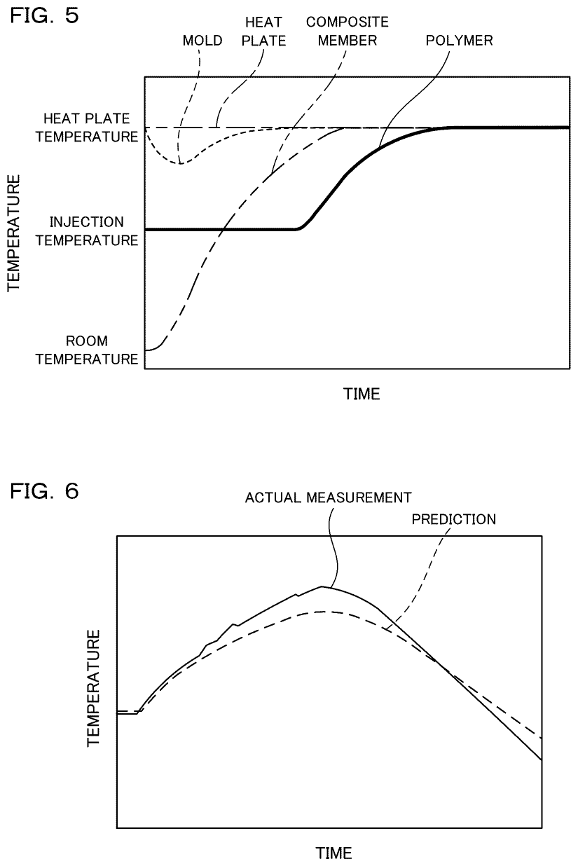

As illustrated in FIG. 5, the temperature of the heat plate 11 is set constant. The temperature of the mold 10 is set to the same temperature as that of the heat plate 11. The temperature of the polymer portion 12 at the time of injection is lower than the set temperature of the mold 10. Therefore, the temperature of the mold 10 is temporarily lowered after the raw material polymer is injected. Thereafter, the temperature of the mold 10 rises and becomes the same as the temperature of the heat plate 11. The temperature of the polymer portion 12 rises after being injected into the mold 10, and becomes the same as the temperature of the mold 10 when a sufficient time elapses. The temperatures of the inner joint member 14 and the outer joint member 13 may be the same as the outside air temperature or may be preheated in a state before being arranged in the mold 10. When the temperature is the same as the outside air temperature, the temperature may be lowered to about 0° C. in winter, for example. In the case of being preheated, the temperature may be raised to any temperature, for example, to around 100° C. After the inner joint member 14 and the outer joint member 13 are arranged in the mold 10, the temperatures of the inner joint member 14 and the outer joint member 13 rise due to thermal conduction from the mold 10, and after the raw material polymer is injected, the temperatures further rise, and become the same as the temperature of the mold 10 when a sufficient time elapses.

As illustrated in FIG. 5, a certain period of time is required until the temperature of the polymer portion 12 reaches the temperatures of the heat plate 11 and the mold 10 and reaches an equilibrium state. The following Equation (1) is used to calculate a non-steady process until reaching the equilibrium state. Equation (1) is what is called a non-steady heat conduction equation.

$$\begin{matrix}[ Mathematical Formula 1 ] & \\ \frac{\partial T}{\partial t} = α \times \frac{{\partial }^{2} T}{\partial {x}^{2}} & ( 1 )\end{matrix}$$

where
t: Time variable
x: Position variable
T: Temperature
α: Thermal diffusivity

The thermal diffusivity α is expressed by the following Equation (2).

$$\begin{matrix}[ Mathematical Formula 2 ] & \\ α = \frac{λ}{ρ \times C} & ( 2 )\end{matrix}$$

where
λ: Thermal conductivity
ρ: Density
C: Specific heat

The thermal diffusivity α and the thermal conductivity λ correspond to the thermal diffusivity characteristic TS. However, the thermal diffusivity characteristic TS is not limited to the thermal diffusivity α and the thermal conductivity λ.

When the thermal diffusivity α is known, the temperature of the polymer portion 12 can be calculated by the non-steady heat conduction equation (1) directly using the thermal diffusivity α. When the thermal diffusivity α is not known, the thermal diffusivity α is calculated from Equation (2) using the thermal conductivity λ, the density ρ, and the specific heat C, and the temperature of the polymer portion 12 can be calculated from Equation (1) using the calculated α.

However, in a case where a value described as the thermal diffusivity α of rubber in general Japanese Industrial Standards (JIS) or the like is used in calculating the temperature of the polymer portion 12 using Equation (1), or in a case where a value described as the thermal conductivity λ in JIS or the like is used in a case of using Equation (2), there was a problem that, as illustrated in FIG. 6, a value when the temperature of the polymer portion 12 of the target work model WM arranged in the mold 10 is actually measured did not match, with sufficient accuracy, a value when the temperature of the polymer portion 12 is predicted based on Equation (1). As a result of intensive studies by the inventors, it has been found that this is because the value of the thermal diffusivity α or the thermal conductivity λ of the raw material polymer of the polymer portion 12 described in JIS or the like is different from the actual value of the thermal diffusivity α or the thermal conductivity λ of the polymer portion 12.

Therefore, by actually measuring the temperature of the polymer portion 12 of the target work model WM that is the measurement target and identifying the thermal diffusivity α or the thermal conductivity λ of the polymer portion 12, the thermal diffusivity α or the thermal conductivity λ of the polymer portion 12 of the target work model WM that is the measurement target was obtained.

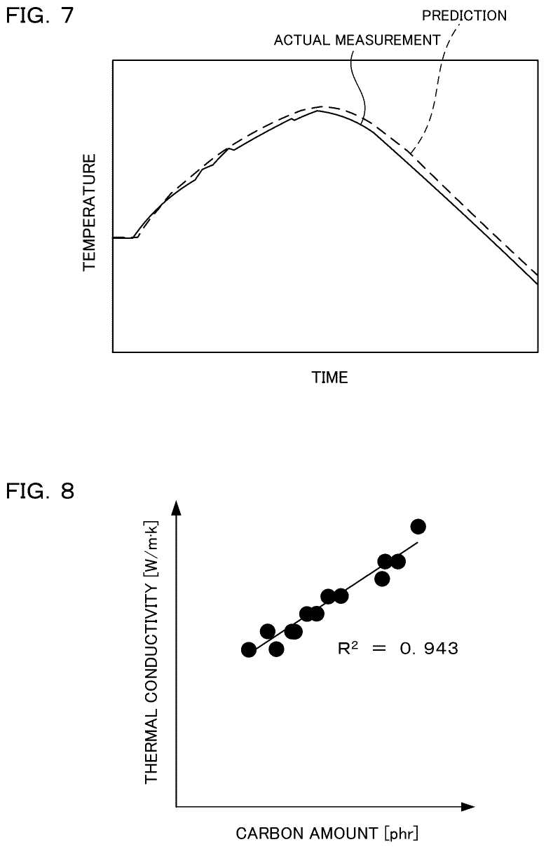

FIG. 7 illustrates the actual measurement value of the temperature of the polymer portion 12 of the target work model WM and the temperature calculated using the actual measurement value of the thermal diffusivity α or the thermal conductivity λ obtained as described above. Then, it has been found that the actual measurement value and the prediction value of the temperature of the polymer portion 12 match each other with high accuracy.

However, the thermal diffusivity α or the thermal conductivity λ of the polymer portion 12 constituting the target work model WM changes depending on the type of the raw material polymer of the polymer portion 12, the formulation amount or the type of the additive material added to the raw material polymer, and the like. Therefore, it is complicated to actually measure the thermal diffusivity α or the thermal conductivity λ for all the polymer portions 12 of different formulations.

Therefore, the inventors have focused on carbon black among the additive materials added to the polymer portion 12. The rubber constituting the polymer portion 12 is a material to which heat is hardly transferred. On the other hand, carbon black is a material to which heat is easily transferred. Therefore, it is considered that the thermal diffusivity α or the thermal conductivity λ of the polymer portion 12 is highly related to the mass ratio of carbon black to the raw material polymer constituting the polymer portion 12. Examples of the carbon black include furnace black, acetylene black, and Ketjen black.

FIG. 8 illustrates the relation between the mass ratio of carbon black to the raw material polymer and the thermal conductivity λ of the polymer portion 12 when experiments are conducted with mass ratios of different carbon black using a plurality of types of raw material polymers. In FIG. 8, the mass ratio of carbon black to the raw material polymer was parts by mass. However, the mass ratio of carbon black to the raw material polymer may be mass %.

As illustrated in FIG. 8, the thermal conductivity λ of the polymer portion 12 has a linear relation with respect to the mass ratio of carbon black to the raw material polymer. It has been found that the thermal conductivity λ of the polymer portion 12 has a relation independent of the type of the raw material polymer. This enables the thermal conductivity λ of the polymer portion 12 to be accurately predicted based on the formulation amount of carbon black regardless of the type of the raw material polymer. Since the thermal diffusivity α can be calculated from the thermal conductivity λ using Equation (2), the thermal diffusivity α can also be accurately predicted.

### 3-2. Contact Heat Transfer Coefficient CH

As illustrated in FIG. 2, there is a case where an outer gap 15A is formed between the inner surface of the mold 10 and the outer joint member 13 in a state where the outer joint member 13 is arranged in the mold 10. In this case, heat is transferred from the inner surface of the mold 10 to the outer joint member 13 via the air in the outer gap 15A. In the heat transfer analysis processing S1, on an assumption of a state where the inner surface of the mold 10 and the outer joint member 13 are in contact with each other, surfaces of the mold 10 and the outer joint member 13 for which the outer gap 15A is considered are selected, and the contact heat transfer coefficient CH is set corresponding to the size of the outer gap 15A. As the contact heat transfer coefficient CH, one in which temperature behavior when the size of the outer gap 15A is changed is measured in an experiment using a test piece and a value that can reproduce each temperature behavior is obtained by analysis is adopted.

There is a case where an inner gap 15B is formed between the inner surface of the mold 10 and the inner joint member 14 in a state where the inner joint member 14 is arranged in the mold 10. In this case, heat is transferred from the inner surface of the mold 10 to the inner joint member 14 via the air in the inner gap 15B. In the heat transfer analysis processing S1, on an assumption of a state where the inner surface of the mold 10 and the inner joint member 14 are in contact with each other, surfaces of the mold 10 and the inner joint member 14 for which the inner gap 15B is considered are selected, and the contact heat transfer coefficient CH is set corresponding to the size of the inner gap 15B. As the contact heat transfer coefficient CH, one in which temperature behavior when the size of the inner gap 15B is changed is measured in an experiment using a test piece and a value that can reproduce each temperature behavior is obtained by analysis is adopted.

As described above, the storage unit 2 stores the contact heat transfer coefficient CH in the gaps 15A and 15B between the forming mold model MM and the metal fitting in a state where the target work model WM is arranged in the forming mold model MM.

In the heat transfer analysis processing S1, the analysis unit 32 is configured to perform the heat transfer analysis using the polymer thermal diffusivity determined by the polymer thermal diffusivity determination unit 31, the temperature condition TC stored in the storage unit 2, and the contact heat transfer coefficient CH, in a state where the target work model WM is arranged in the forming mold model MM. This enables the temperature of the polymer portion 12 to be accurately predicted.

### 3-3. Air Heat Transfer Coefficient AC

Since the temperature of the polymer portion 12 of the target work model WM does not decrease immediately also after the target work model WM is demolded from the forming mold model MM, the crosslinking reaction progresses in the polymer portion 12. Therefore, in the present embodiment, in order to predict the temperature of the polymer portion 12 also after the target work model WM is demolded from the forming mold model MM, the condition input unit 30 receives the outside air condition OC including the air temperature around the target work model WM demolded from the forming mold model MM. Furthermore, the storage unit 2 stores the air heat transfer coefficient AC, which is the heat transfer coefficient of the air around the target work model WM demolded from the forming mold model MM.

The analysis unit 32 of the present embodiment performs heat transfer analysis using the polymer thermal diffusivity determined by the polymer thermal diffusivity determination unit 31, the air heat transfer coefficient AC stored in the storage unit 2, and the outside air condition OC, in a state where the polymer portion 12 of the target work model WM is caused to undergo the crosslinking reaction in the forming mold model MM and then the target work model WM is demolded from the forming mold model MM. This enables the temperature of the polymer portion 12 to be accurately predicted in a state after the target work model WM is demolded from the forming mold model MM.

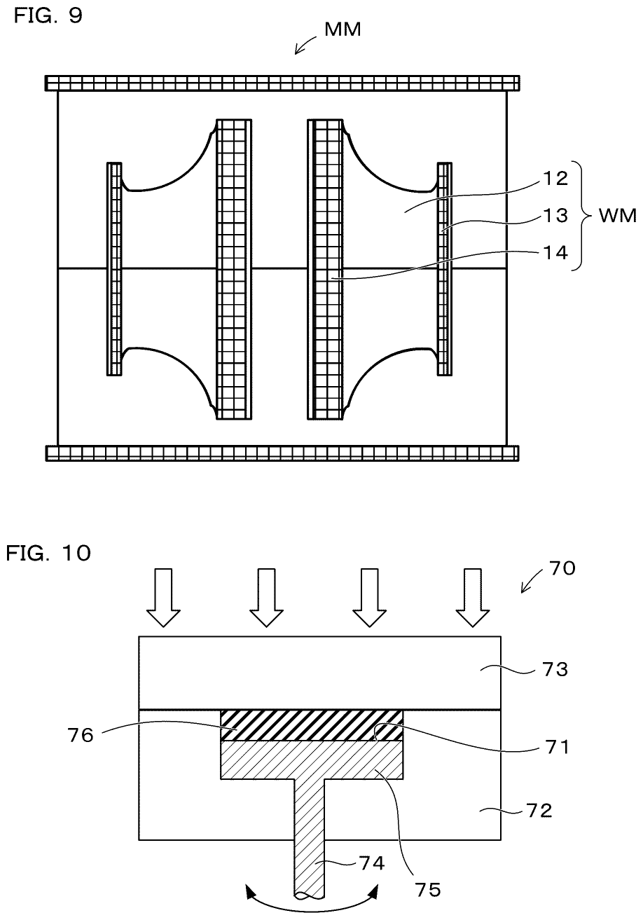

As illustrated in FIG. 9, an analysis mesh is created for a state where the target work model WM is arranged in the forming mold model MM. The size of the analysis mesh may be determined in advance or may be input from the condition input unit 30. In creating the analysis mesh, a characteristic (e.g., symmetry) of the shapes of the forming mold model MM and the target work model WM are considered. As a mesh type, an 8-node solid element was adopted. However, any model can be adopted as a mesh type, and for example, a four-node solid element may be used.

Use of the analysis mesh enables the temperature at each node of the target work model WM to be predicted. That is, the temperature distribution of the polymer portion 12 of the target work model WM can be predicted. In particular, the temperature of each node of the polymer portion 12 at each time can be predicted.

### 4. Crosslinking Reaction Analysis Processing S2

Next, the crosslinking reaction analysis processing S2 will be described. Regarding the crosslinking reaction analysis processing S2, the reaction rate will be described first. Next, a conventional technique will be described, and then problems of the conventional technique will be described. Thereafter, the crosslinking reaction analysis processing of the present embodiment will be described.

### 4-1. About Reaction Rate

The reaction rate of the crosslinking reaction is measured as follows. As illustrated in FIG. 10, regarding the reaction rate of the rubber according to the present embodiment, the reaction rate (vulcanization degree) is calculated from the relation between the obtained torque and the reaction time using a vulcanization tester 70 (an example of a crosslinking reaction characteristic tester) in accordance with JIS K6300-2. As the vulcanization tester 70, for example, a curelastometer (registered trademark) can be used. When the vibration characteristic is output in consideration of the reaction rate (vulcanization degree), the reaction behavior (vulcanization behavior) under a desired vibration condition can be measured using, for example, a rubber process analyzer (RPA). The RPA is configured to be able to set a frequency and an amplitude in a wide range. However, the raw material polymer is not limited to the rubber described in JIS K6300-2. Note that JIS K6300-2 is created based on ISO6502:1999, and some specified items not included in corresponding international standards are added. Furthermore, some of the corresponding international standards have been modified and deleted.

As illustrated in FIG. 10, in the vulcanization tester 70, a disk 75 attached to the tip end of a rotor 74 is arranged between a lower die 72 and an upper die 73 having a cavity 71. The lower die 72 and the upper die 73 can be set to predetermined temperatures. The rotor 74 has a twisting angle selectable between +3° and −3° or between +1° and −1°. A measurement sample 76 is arranged between the cavity 71 and the disk 75, and is subjected to a crosslinking reaction while rotational torque is applied from the disk 75 at a constant temperature, and the progress of the crosslinking reaction is detected from a change in the torque.

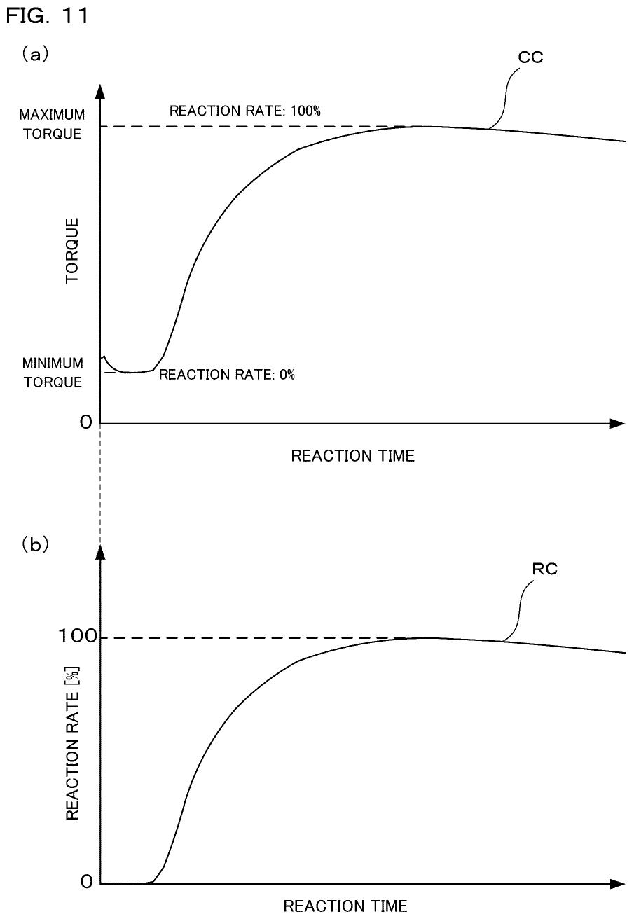

FIG. 11 shows an example of the measurement result. FIG. 11(a) is a graph (crosslinking reaction curve CC) of the change amount in torque with respect to the reaction time at a predetermined reaction temperature. On the other hand, the reaction rate is defined such that the minimum torque is a reaction rate of 0% and the maximum torque is a reaction rate of 100%. FIG. 11(b) shows a graph (reference reaction curve RC) showing a change in the reaction rate with respect to the reaction time at a predetermined reaction temperature. In the case of the rubber according to the present embodiment, the reaction rate is called a vulcanization degree. The vulcanization degree is defined as the degree of vulcanization when the physical properties (elastic modulus, elongation, tensile strength, hardness, and the like) of vulcanized rubber is used as an index. Note that in FIG. 11(a), the region from reaction time 0 to the reaction time showing the minimum torque is a plastic region in which the crosslinking reaction of the polymer portion 12 is unreacted. Therefore, in FIG. 11(b), the reaction rate is 0% in this region.

### 4-2. Calculation Method of Reaction Rate

Next, a calculation method of the reaction rate will be described. In Non Patent Literature 1 described above, the equivalent reaction amount of the crosslinking reaction is estimated using Equation (4) related to the equivalent reaction amount obtained by modifying Arrhenius Equation (3). Note that Equation (4) related to the equivalent reaction amount expresses how many times the reaction amount at a reaction temperature T and a reaction time t greater than the reaction amount at a certain reference temperature T₀ and a reference time t₀. Note that in a vulcanization reaction of rubber, Equation (4) is called an equation of equivalent vulcanization. Use of this equation of equivalent vulcanization enables the reaction rate in a reaction return period described later to be predicted. As described in detail later, in the present embodiment, the prediction accuracy in the reaction return period can be further improved as compared with the case of simply using the equation of equivalent vulcanization. Note that the reaction rate in the reaction return period cannot be predicted by an equation (e.g., the reaction speed equation of Kamal) used for predicting the behavior of the thermosetting reaction of rubber, epoxy, or the like in general analysis software.

$$\begin{matrix}[ Mathematical Formula 3 ] & \\ k = A \times exp ( - \frac{E}{R \times T} ) & ( 3 )\end{matrix}$$

where
k: Reaction speed
A: Frequency factor
E: Activation energy
R: Gas constant
T: Reaction temperature

$$\begin{matrix}[ Mathematical Formula 4 ] & \\ U = exp \{ - \frac{E}{R} \times ( \frac{1}{T} - \frac{1}{{T}_{0}} ) \} \times t & ( 4 )\end{matrix}$$

where
U: Equivalent reaction amount
E: Activation energy
R: Gas constant
T: Reaction temperature
T₀: Reference temperature
t: Reaction time

Equation (4) of the equivalent reaction amount is derived from Arrhenius Equation (3) by modifying the equation as follows.

A reaction amount Z of the crosslinking reaction at the reaction time t is expressed by Equation (5).

$$\begin{matrix}[ Mathematical Formula 5 ] & \\ Z = k \times t & ( 5 )\end{matrix}$$

where
Z: Reaction amount
t: Reaction time

The reaction amount Z is as the following Equation (6) from Arrhenius Equation (3) and the above Equation (5).

$$\begin{matrix}[ Mathematical Formula 6 ] & \\ Z = A \times exp ( - \frac{E}{R \times T} ) \times t & ( 6 )\end{matrix}$$

Here, considering a reaction amount Z₀ at the reaction time t₀ at the reference reaction temperature T₀, Z₀ is expressed by the following Equation (7).

$$\begin{matrix}[ Mathematical Formula 7 ] & \\ {Z}_{0} = A \times exp ( - \frac{E}{R \times {T}_{0}} ) \times {t}_{0} & ( 7 )\end{matrix}$$

A ratio U of the reaction amount Z at the reaction temperature T and the reaction time t to the reaction amount Z₀ at the reaction temperature T₀ and the reaction time t₀ is expressed by the following Equation (8).

$$\begin{matrix}[ Mathematical Formula 8 ] & \\ U = \frac{Z}{{Z}_{0}} & ( 8 )\end{matrix}$$

By substituting Equation (6) and Equation (7) into Equation (8), Equation (9) is obtained for the ratio U.

$$\begin{matrix}[ Mathematical Formula 9 ] & \\ U = exp \{ - \frac{E}{R} \times ( \frac{1}{T} - \frac{1}{{T}_{0}} ) \} \times \frac{t}{{t}_{0}} & ( 9 )\end{matrix}$$

In Equation (9), when t₀=1, Equation (9) is expressed as Equation (4) described above.

When the reaction temperature T changes from moment to moment, an equivalent reaction amount increase amount in a minute time at the target time is calculated based on the temperature at the target time, the above Equation (4), and the slope coefficient SC, an equivalent reaction amount integration value from the start of the crosslinking reaction to the target time is calculated based on the equivalent reaction amount increase amount, and a reaction rate at the target time is calculated based on the equivalent reaction amount integration value from the start of the crosslinking reaction to the target time.

Based on Equation (4), an equivalent reaction amount increase amount ΔUᵢ at a minute time Δt of the target time is expressed by the following Equation (10).

$$\begin{matrix}[ Mathematical Formula 10 ] & \\ Δ {U}_{i} = exp \{ - \frac{E}{R} \times ( \frac{1}{T} - \frac{1}{{T}_{0}} ) \} \times Δ t & ( 10 )\end{matrix}$$

where
ΔUᵢ: Equivalent reaction amount increase amount in minute time at target time
Δt: Minute time of target time

When the reaction temperature T changes from moment to moment, the equivalent reaction amount U can be obtained by accumulating for each unit time as in the following Equation (11).

$$\begin{matrix}[ Mathematical Formula 11 ] & \\ U = {\sum }_{i = 1}^{n} exp \{ - \frac{E}{R} \times ( \frac{1}{T} - \frac{1}{{T}_{0}} ) \} \times Δ t & ( 11 )\end{matrix}$$

where
U: Equivalent reaction amount
E: Activation energy
R: Gas constant
T: Reaction temperature
T₀: Reference temperature
Δt: Minute time of target time

The above Equation (4) is an equation inside Σ in the above Equation (11), and corresponds to each equivalent reaction amount that is accumulated.

In order to calculate Equation (4) or Equation (11), the value of (−E/R) is required. In the conventional technique, (−E/R) is obtained as follows.

By taking the natural logarithms of both sides of Arrhenius Equation (3), the following Equation (12) is obtained.

$$\begin{matrix}[ Mathematical Formula 12 ] & \\ ln k = ( - \frac{E}{R} ) \times ( \frac{1}{T} ) + ln A & ( 12 )\end{matrix}$$

where
k: Reaction speed
A: Frequency factor
E: Activation energy
R: Gas constant
T: Reaction temperature

Regarding the above reaction amount, it is considered that the following Equation (13) is established between ae reaction time t_{γ} at a predetermined reaction rate γ and a reaction speed k. As the reaction rate γ, for example, any value of 0% to 100% such as 5% or 90% can be appropriately adopted.

$$\begin{matrix}[ Mathematical Formula 13 ] & \\ k = B \times ( \frac{1}{{t}_{α}} ) & ( 13 )\end{matrix}$$

where
B: Arbitrary constant
t_{γ}: Reaction time at predetermined reaction rate γ

By taking the natural logarithms of both sides of Equation (13) and substituting them into Equation (12) to deform the equation, thereby giving Equation (14).

$$\begin{matrix}[ Mathematical Formula 14 ] & \\ ln ( \frac{1}{{t}_{α}} ) = ( - \frac{E}{R} ) \times ( \frac{1}{T} ) + ln A - ln B & ( 14 )\end{matrix}$$

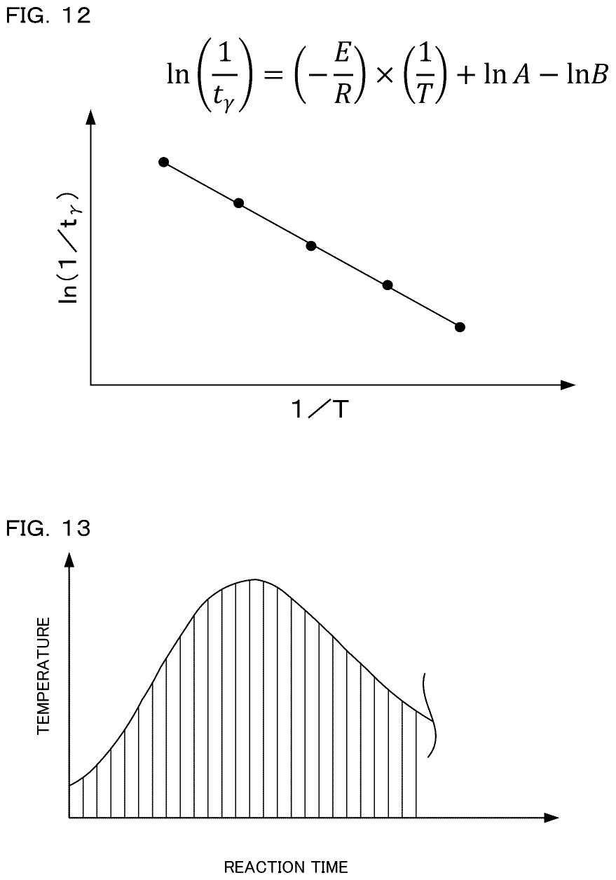

From Equation (14), it is found that when the time at which the reaction rate reaches α % and each measurement temperature are plotted with ln(1/t_{γ}) on the vertical axis and (1/T) on the horizontal axis, approximation to a straight line can be performed, and the slope of this straight line is (−E/R) (see FIG. 12).

In an actual reaction, as illustrated in FIG. 13, the temperature changes with the lapse of the reaction time. In such a case, the equivalent reaction amount is calculated as follows using Equations (4) and (11).

First, as illustrated in FIG. 13, the reaction time is divided at predetermined intervals (e.g., 1 minute). Next, the temperature at each predetermined interval is read.

Next, the equivalent reaction amount per predetermined interval is calculated using Equation (4). In Equation (4), the calculation is performed by substituting the predetermined interval Δt. For example, the calculation can be simplified by setting the predetermined interval as Δt=1.

Next, ΔUᵢ per predetermined interval Δt calculated by Equation (4) is added. A specific calculation method will be described with reference to FIG. 14.

As illustrated in FIG. 14, an equivalent reaction amount Uᵢ₋₁ at a reaction time tᵢ₋₁ and a reaction temperature Tᵢ₋₁ is calculated by Equation (4). Similarly, an equivalent reaction amount Uᵢ at a reaction time t₁ and a reaction temperature Tᵢ is calculated.

Next, Uᵢ₋₁×Δt and Uᵢ×Δt are added. This gives an integration value of the equivalent reaction amount in the reaction times tᵢ₋₁ to tᵢ. Note that in the present embodiment, Δt=½t. Similarly, the equivalent reaction amount U can be obtained by integrating the equivalent reaction amounts ΔUᵢ per predetermined time from t=1 to t=n in accordance with Equation (11).

Subsequently, a method of calculating the reaction rate from the equivalent reaction amount U will be described. First, the reaction amount Z at the reaction temperature T₀ and the reaction time t is represented by Equation (5). Next, from Equation (5), the reaction amount Z₀ at the reaction temperature T₀ and the reaction time t₀ is represented by the following Equation (15). Here, since the reaction temperatures of Equation (5) and Equation (15) are the same value T₀, each k is the same value from Equation (3).

$$\begin{matrix}[ Mathematical Formula 15 ] & \\ {Z}_{0} = k \times {t}_{0} & ( 15 )\end{matrix}$$

When the above Equations (5) and (15) are substituted into the above Equation (8), the reaction speed k is reduced to obtain the following Equation (16).

$$\begin{matrix}[ Mathematical Formula 16 ] & \\ U = \frac{t}{{t}_{0}} & ( 16 )\end{matrix}$$

In Equation (16), when t₀=1, Equation (16) is expressed as the following Equation (17).

$$\begin{matrix}[ Mathematical Formula 17 ] & \\ U = t & ( 17 )\end{matrix}$$

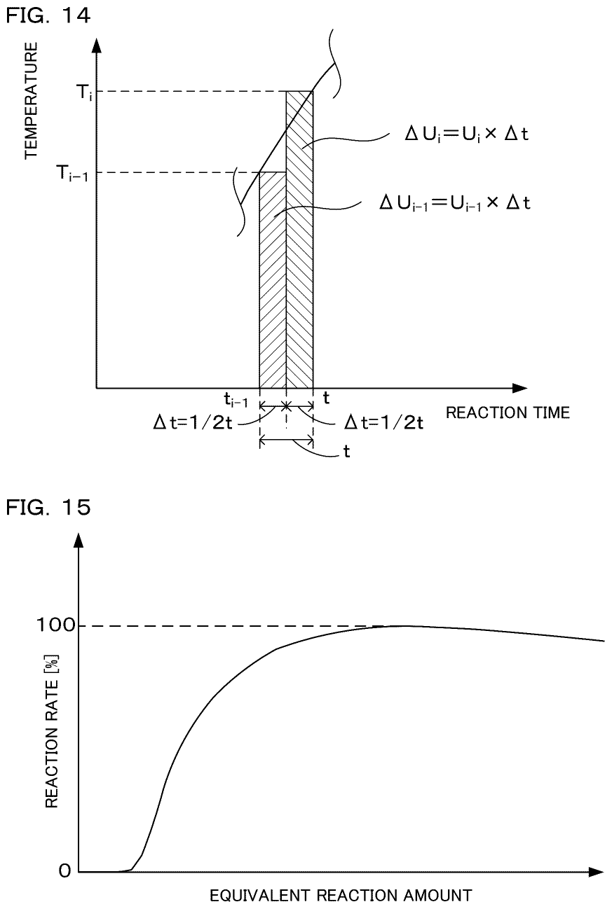

According to Equation (17), when t₀=1, the reaction time t can be read as the equivalent reaction amount U. By this, in the graph showing the change in the reaction rate with respect to the reaction time at the reaction temperature T₀ shown in FIG. 11(b), the reaction time t can be read as the equivalent reaction amount U. FIG. 15 is a graph in which the vertical axis represents the reaction rate and the horizontal axis represents the equivalent reaction amount. Based on this graph, the reaction rate can be calculated from the equivalent reaction amount U obtained by calculation.

### 4-3. Problem of Reaction Rate Calculation Method

However, when the reaction rate is estimated based on the above method, there is a problem that a difference from the actual measurement value is large and sufficient accuracy cannot be obtained.

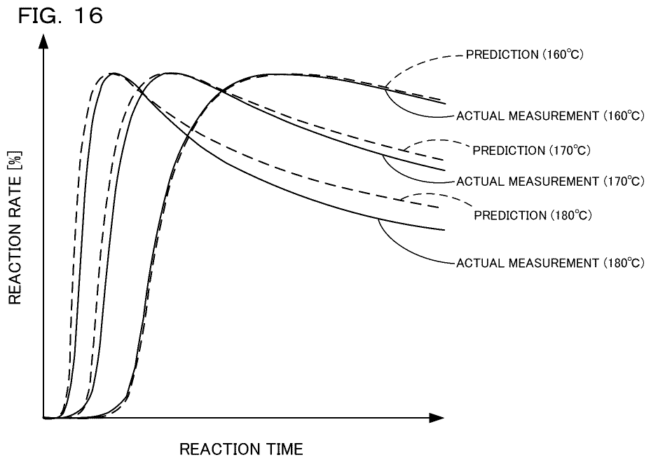

FIG. 16 illustrates a change in the reaction rate with respect to the reaction time. Since the reference temperature was set to 160° C. when Equation (4) was used, the actual measurement value and the prediction value accurately match in the case of the reaction temperature of 160° C.

However, at the reaction temperatures of 170° C. and 180° C., the actual measurement value and the prediction value do not sufficiently match each other. In general, the prediction value is larger than the actual measurement value. Note that the maximum value of the reaction rate matches each other because the maximum value of the reaction rate was defined as 100% as described above.

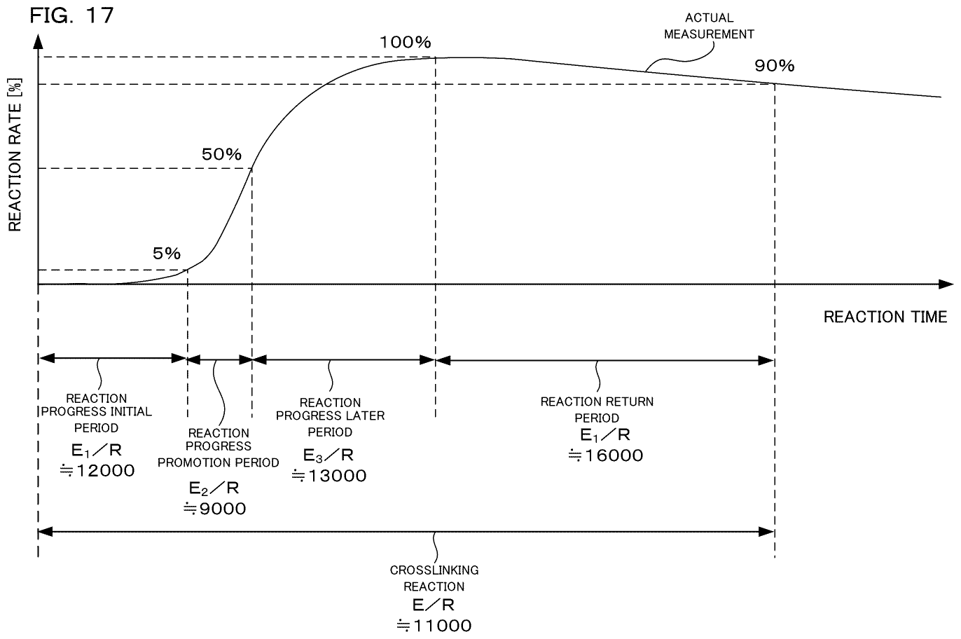

The reason why the actual measurement value and the prediction value do not sufficiently match in the conventional technique will be described with reference to FIG. 17. FIG. 17 shows a graph showing a change in the reaction rate with respect to the reaction time. The crosslinking reaction according to the present embodiment progresses through the following process.

The reaction rate gradually increases in a reaction progress initial period in which the reaction started. When a reaction progress promotion period is reached with a lapse of time to some extent, the reaction rate rapidly increases. Thereafter, in a reaction progress later period, the increase amount of the reaction rate becomes gentle, and the reaction rate reaches the maximum value. After the reaction rate reaches the maximum value, the process enters a reaction return period in which a return reaction progresses. In this reaction return period, the reaction rate gradually decreases.

In the technique described above, the reaction progress initial period to the reaction return period is regarded as one crosslinking reaction. Therefore, the reaction rate was predicted using one activation energy E. As a result, it is considered that the actual measurement value of the reaction rate does not sufficiently match the prediction value.

### 4-4. Crosslinking Reaction Analysis Processing of Present Embodiment

In the present embodiment, a plurality of activation energies are calculated corresponding to the reaction progress state, and the equivalent reaction amount is predicted based on the obtained activation energies. Based on this idea, Equation (4) was transformed into the following Equation (18).

$$\begin{matrix}[ Mathematical Formula 18 ] & \\ Δ {U}_{i} = exp \{ - \frac{{E}_{N}}{R} \times ( \frac{1}{T} - \frac{1}{{T}_{0}} ) \} \times Δ t & ( 18 )\end{matrix}$$

where
E_{N}: Activation energy for each stage of reaction progression divided into N stages

FIG. 17 shows an example of dividing the reaction progress state into four stages of the reaction progress initial period, the reaction progress promotion period, the reaction progress later period, and the reaction return period. However, the reaction progress state may be divided into any stages of two to three or five or more. The entire period including the reaction progress period and the reaction return period may be divided at equal intervals. As an index for division into equal intervals, for example, the degree of progress of the crosslinking reaction may be divided into 5% based on the degree of progress of the crosslinking reaction over the entire period. However, the index of the division is not limited to 5%, and may be 1% to 4% or 6% or more.

In the present embodiment, the reaction rate of 0% to 5% was defined as the reaction progress initial period, the reaction rate of 5% to 50% was defined as the reaction progress promotion period, the reaction rate of 50% to 100% was defined as the reaction progress later period, and a range in which the reaction rate decreased from 100% to 90% in a stage after the reaction progress later period was defined as the reaction return period.

In each reaction stage, the value of the term (E_{N}/R) including the activation energy E_{N} was obtained using the above Equation (14). As shown in FIG. 17, (E₁/R) at the reaction progress initial period (reaction rate is 0% to 5%) was about 12000, (E₂/R) at the reaction progress promotion period (reaction rate is 5% to 50%) was about 9000, (E₃/R) at the reaction progress later period (reaction rate is 50% to 100%) was about 13000, and (E₄/R) at the reaction return period (reaction rate is 100% to 90%) was about 16000. However, the numerical values of (E_{N}/R) are not limited to the above values. Note that (E/R) when the reaction progress initial period to the reaction return period was regarded as one reaction stage was 11000.

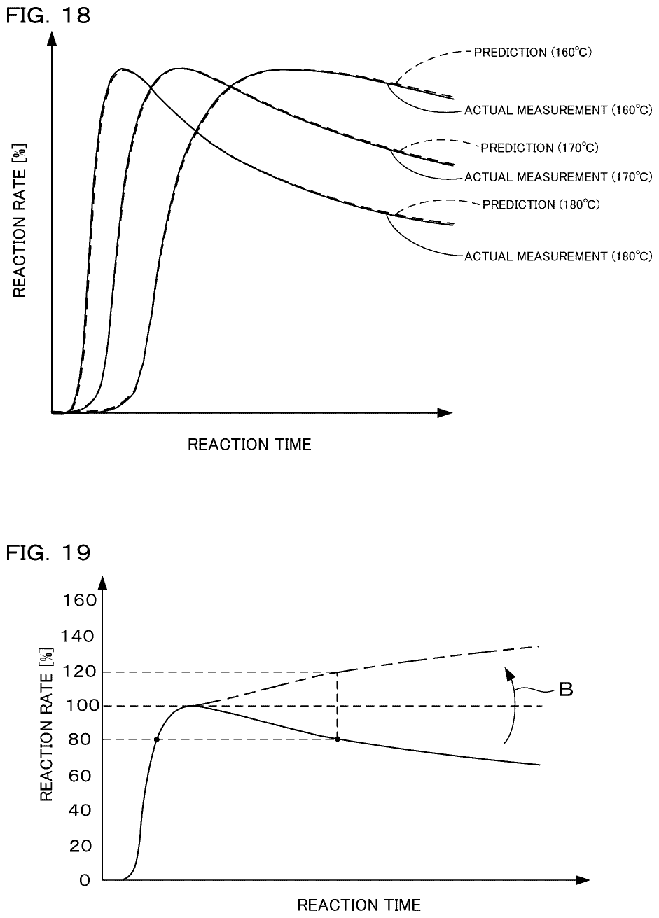

FIG. 18 illustrates, together with the actual measurement values, the result of predicting the reaction rate using the activation energies E₁ to E₄ obtained as described above. The actual measurement value and the prediction value not only matched at 160° C., which is the reference temperature, but also accurately matched also at the reaction temperatures of 170° C. and 180° C. This can accurately predict the reaction rate.

In the present embodiment, the slope coefficient SC is set to different values between the reaction progress period until the reaction rate of the crosslinking reaction reaches the peak and the reaction return period when the reaction rate exceeds the peak. In the present embodiment, the slope coefficient SC is set to a smaller value in the reaction return period than in the reaction progress period.

In the present embodiment, the slope coefficient SC is set to different values respectively at the reaction progress initial period, the reaction progress promotion period, the reaction progress later period, and the reaction return period. In the present embodiment, the slope coefficient SC in the reaction promotion period is set to a value larger than those of the reaction progress initial period and the reaction progress later period.

The slope coefficient SC is set to a value corresponding to each division when the entire period of the crosslinking reaction is divided at equal intervals.

### 4-5. Method of Expressing Reaction Rate of Crosslinking Reaction

A method of expressing the reaction rate of the crosslinking reaction in the present embodiment will be described with reference to FIG. 19. FIG. 19 is a graph showing a change in the reaction rate of the crosslinking reaction with respect to the reaction time. The graph indicated by the solid line is an actual measurement value of the reaction rate.

After the reaction rate of the crosslinking reaction indicated by the solid line reaches the maximum value, the reaction rate gradually decreases as the return reaction progresses. Therefore, for example, there are a point at which the reaction rate becomes 80% before the reaction rate reaches the maximum value and a point at which the reaction rate becomes 80% after the reaction rate reaches the maximum value. In this case, when only the numbers of the reaction rate are simply compared, the numerical value of 80% before the reaction rate reaches the maximum value cannot be distinguished from the numerical value of 80% after the reaction rate reaches the maximum value.

Therefore, in the present embodiment, the reaction rate of a crosslinking reaction during a reaction progress period until the reaction rate reaches a peak is set to 0% at a beginning of the crosslinking reaction, and is defined in a range of 0% to 100% as a degree of increase in the reaction rate, and in a reaction return period when the reaction rate exceeds the peak, the reaction rate of a crosslinking reaction is defined as a value obtained by adding 100% to a degree of decrease in reaction rate from a reaction rate peak.

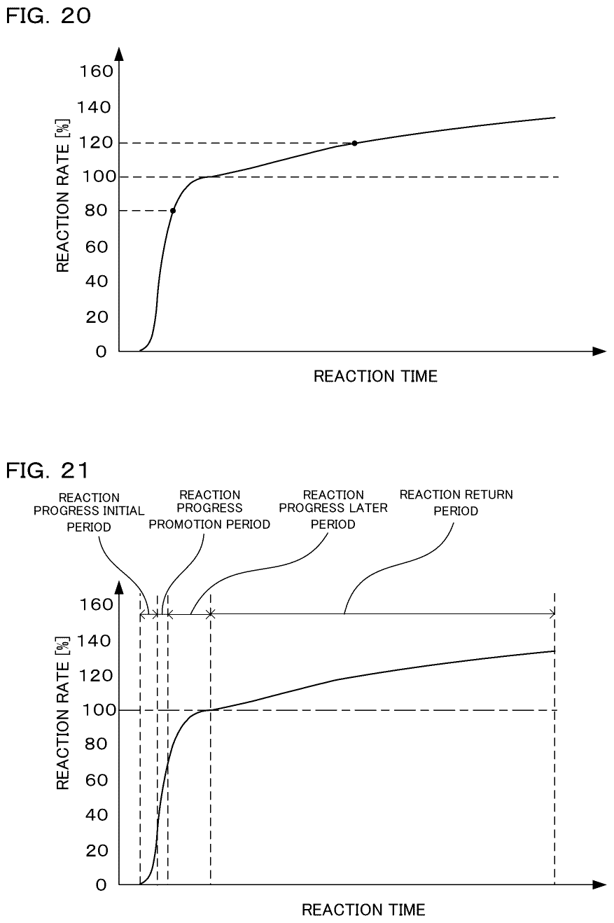

With reference to the graph of FIG. 19, with a straight line extending parallel to the horizontal axis from a point where the reaction rate is 100% as a symmetry axis, the graph of the reaction return period in which the reaction rate exceeds the peak is inverted as indicated by an arrow B. Thus, the point at which the reaction rate is 80% after the reaction rate reaches the maximum value can be expressed as a reaction rate of 120%. As a result, the reaction rate before the reaction rate reaches the maximum value and the reaction rate after the reaction rate reaches the maximum value can be clearly distinguished (see FIG. 20).

### 4-6. Determination Method of Value Calculated by Simulation

Next, a determination method of a value calculated by simulation will be described. In the present embodiment, the slope coefficient SC and the reaction rate are determined using one or both of a database and a function. Use of the database can improve the accuracy of an estimation value. On the other hand, use of the function can improve the calculation speed.

### (1) Method Using Database

As illustrated in FIG. 1, the storage unit 2 stores the first relation data map DM1 defining a correspondence relation between the reaction rate of the crosslinking reaction of the polymer portion 12 and the slope coefficient SC. The reaction rate calculation processing unit 41 determines the slope coefficient SC using the reaction rate at the previous time and the first relation data map DM1.

The storage unit 2 stores the second relation data map DM2 defining a correspondence relation between the equivalent reaction amount integration value and the reaction rate at a target time. The reaction rate calculation processing unit 41 determines the reaction rate at the target time using the equivalent reaction amount integration value at the target time and the second relation data map DM2.

The second relation data map DM2 is set by the reference reaction curve RC based on the relation between the crosslinking reaction time and the torque generated in the test target polymer material the reference reaction temperature obtained by measuring the test target polymer material, which corresponds to the polymer portion 12 of the target work model WM, with the vulcanization tester 70.

### (2) Method Using Function

As illustrated in FIG. 1, the storage unit 2 stores the first function F1 defining a correspondence relation between the reaction rate of the crosslinking reaction of the polymer portion 12 and the slope coefficient SC. The reaction rate calculation processing unit 41 determines the slope coefficient SC using the reaction rate at the previous time and the first function F1.

The first function F1 is set to a different function corresponding to a plurality of reaction rate divisions set corresponding to the degree of progress of the crosslinking reaction in the polymer portion 12. The number of divisions of the degree of progress of the crosslinking reaction is arbitrary, and may be divided into one or a plurality of two or more divisions. For the function, any function such as an nth-order function including linear complement or a spline curve can be appropriately selected. In the present embodiment, for example, a sixth-order function is preferably used. Note that the number of divisions of the first function F1 can be set independently of the division for obtaining the value of the activation energy E_{N} for each of the progress stages of the reaction divided into the N stages.

The storage unit 2 stores the second function F2 defining a correspondence relation between the equivalent reaction amount integration value and the reaction rate at a target time. The reaction rate calculation processing unit 41 determines the reaction rate at the target time using the equivalent reaction amount integration value at the target time and the second function F2.

The second function F2 is set by the reference reaction curve RC based on the relation between the crosslinking reaction time and the torque generated in the test target polymer material the reference reaction temperature obtained by measuring the test target polymer material, which corresponds to the polymer portion 12 of the target work model WM, with the vulcanization tester 70.

The second function F2 is set to a different function in accordance with a plurality of reaction rate divisions set corresponding to the degree of progress of the crosslinking reaction of the polymer portion 12. The number of divisions of the degree of progress of the crosslinking reaction is arbitrary, and may be divided into one or a plurality of two or more divisions. For the function, any function such as an nth-order function including linear complement or a spline curve can be appropriately selected. In the present embodiment, for example, a sixth-order function is preferably used. Note that the number of divisions of the second function F2 can be set independently of the division for obtaining the value of the activation energy E_{N} for each of the progress stages of the reaction divided into the N stages.

FIG. 21 exemplifies a function when the degree of progress of the crosslinking reaction is divided into four for the second function F2. In the present embodiment, the degree of progress of the crosslinking reaction is divided into the reaction progress initial period, the reaction progress promotion period, the reaction progress later period, and the reaction return period. In each division, the reaction rate and the reaction time are approximated by functions different from each other.

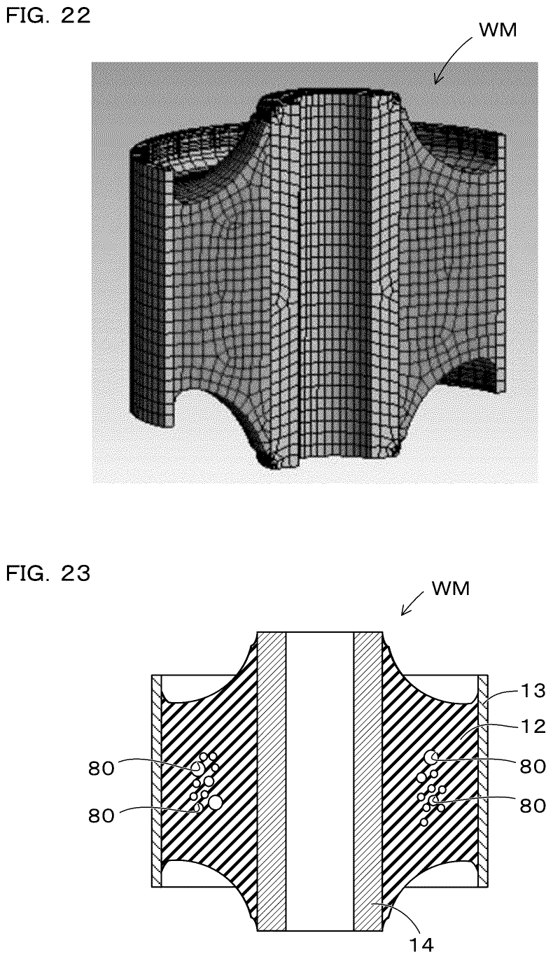

Next, an analysis mesh of the target work model WM is created. The size of the analysis mesh may be determined in advance or may be input from the condition input unit 30. In creating the analysis mesh, an analysis mesh as illustrated in FIG. 22 is created in consideration of the characteristic (e.g., symmetry) of the shape of the target work model WM. As a mesh type, an 8-node solid element was adopted. However, any model can be adopted as a mesh type, and for example, a four-node solid element may be used.

Using the analysis mesh, it is possible to calculate the reaction rate of each node from the temperature of each node at each time of only the polymer portion 12 of the target work model WM.

### 5. Structural Analysis Processing S3

### 5-1. Initial Processing

Next, the structural analysis processing S3 will be described. The structural analysis unit 5 creates an analysis mesh of the target work model WM. The analysis mesh of the target work model WM may be the same as or different from the analysis mesh created in the crosslinking reaction analysis processing S2. In the present embodiment, the analysis mesh used in the structural analysis processing S3 is the same as the analysis mesh used in the crosslinking reaction analysis processing S2.

### 5-2. Acquisition of Temperature and Reaction Rate

Next, the structural analysis unit 5 configures to acquire an analysis result of the temperature distribution of the polymer portion 12 of the target work model WM analyzed by the heat transfer analysis unit 3. The structural analysis unit 5 acquires an analysis result of the reaction rate of the polymer portion 12 of the target work model WM analyzed by the crosslinking reaction analysis unit 4.

### 5-3. Elastic Modulus Prediction

Next, the structural analysis unit 5 configures to calculate the elastic modulus of each node at each time of the polymer portion 12 of the target work model WM. First, a test piece having a reaction rate of crosslinking reaction of 100% is created, and the elastic modulus of this test piece is actually measured. Next, a correction coefficient is calculated based on a quotient in which the torque at the target reaction rate is divided by the torque at the reaction rate of 100%. The torque described above is torque measurable by the vulcanization tester 70. The structural analysis unit 5 calculates the elastic modulus of each node at each time by multiplying the actually measured elastic modulus by the calculated correction coefficient.

### 5-4. Characteristic Prediction

Next, the structural analysis unit 5 configures to predict a characteristic of the target work model WM. The input conditions used for finite element analysis are the temperature of each node at each time, the elastic modulus of each node at each time, and a boundary condition between the outer joint member 13 and the inner joint member 14.

After the input condition is set, force or strain in a direction perpendicular to the axis A of the target work model WM is applied to the target work model WM.

The structural analysis unit 5 configures to output the overall elastic modulus of the target work model WM. In the present embodiment, the elastic modulus with respect to the force statically applied in the direction perpendicular to the axis A of the target work model WM is predicted as the characteristic. However, the characteristic to be predicted is not limited to the elastic modulus with respect to the force or strain in the direction perpendicular to the axis A, and any physical property value such as the elastic modulus with respect to the force vibrating in the direction perpendicular to the axis A, the elastic modulus with respect to the force or strain statically applied in the direction along the axis A, or the elastic modulus with respect to the force vibrating in the direction along the axis A can be predicted as the characteristic.

### 6. Internal Bubble Estimation Processing S4

Next, the internal bubble estimation processing S4 will be described. A rubber product that is an example of the present embodiment is formed by molding and vulcanizing. Therefore, the gas originally dissolved in the rubber or the gas generated by the vulcanization reaction is in a state of being dissolved in the rubber under the high temperature and high pressure conditions of vulcanization. When the mold 10 is opened, the pressure applied to the rubber is reduced, and the solubility of the gas in the rubber is reduced, and therefore, in a state where vulcanization is not sufficiently progressed, internal bubbles 80 may be generated in the polymer portion 12 made of rubber (see FIG. 23).

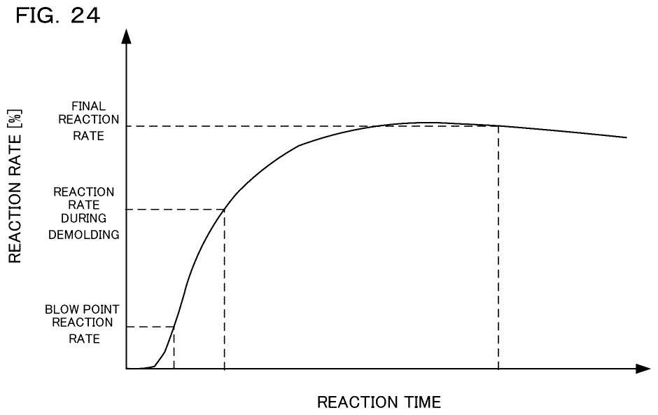

When the vulcanization time is lengthened in a state where the rubber is arranged in the mold 10 and the vulcanization is caused to progress, the internal bubbles 80 are not generated. In vulcanization of an actual product, the total amount of heat received is different due to a difference in the temperature rise history in a point inside of the product having a different distance from the mold 10 that is a heat source, and the vulcanization progress status is different even in the same vulcanization time. Therefore, in the slowest vulcanization part, it is necessary to perform vulcanization by heating in the mold 10 until the time when generation of the internal bubbles 80 is not observed. The vulcanization degree of the rubber in the slowest vulcanization part at the time point when the mold 10 is opened when molding is performed in the vulcanization time until the generation of the internal bubbles 80 is not observed in this slowest vulcanization part is called a blow point vulcanization degree (hereinafter, blow point) (see FIG. 24).

In order to suppress the internal bubbles 80, the heating time in the mold 10 is preferably as long as possible. However, if the heating time in the mold 10 is excessively extended, the manufacturing efficiency of the product decreases. For this reason, in order to determine the vulcanization time of the product, it is important to estimate the blow point described above.

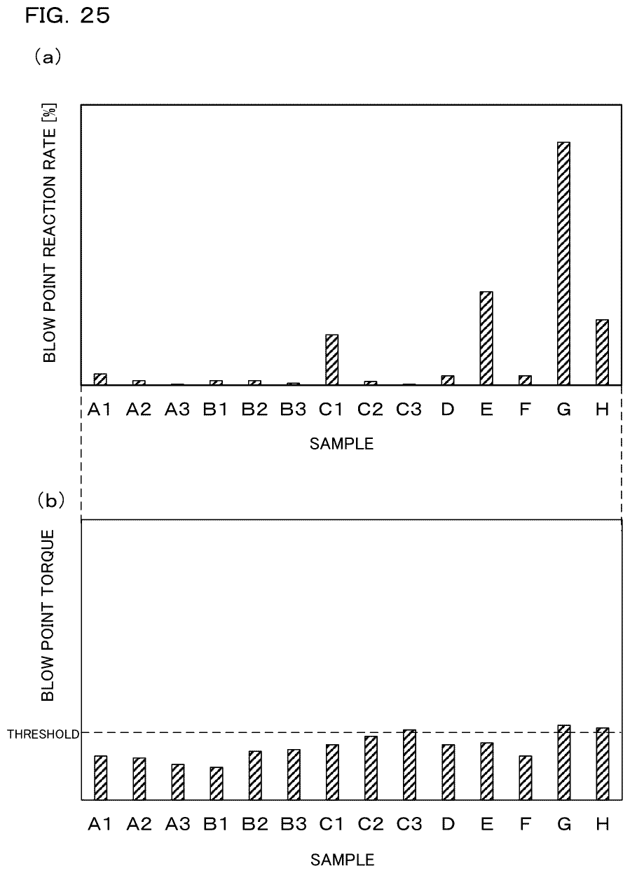

FIG. 25(a) shows a blow point reaction rate when a plurality of types of fillers are added to a plurality of types of polymers in different formulation amounts. The blow point reaction rate is the reaction rate of the crosslinking reaction of the polymer portion 12 at the blow point.

As the raw material polymer, eight types of polymers A to H were used. In samples A1 to A3, the formulation amount of the filler is changed using the raw material polymer A. Similarly, in samples B1 to B3, the formulation amount of the filler is changed with respect to the raw material polymer B, and in samples C1 to C3, the formulation amount of the filler is changed with respect to the raw material polymer C. Since a commercially available product was used as the sample, the type and formulation amount of the filler are not accurately known.

As shown in FIG. 25(a), the blow point reaction rate greatly varies depending on the type of the raw material polymer and the formulation amount of the filler. Therefore, it has been found that it is difficult to predict the blow point of the polymer portion 12 using the blow point reaction rate.

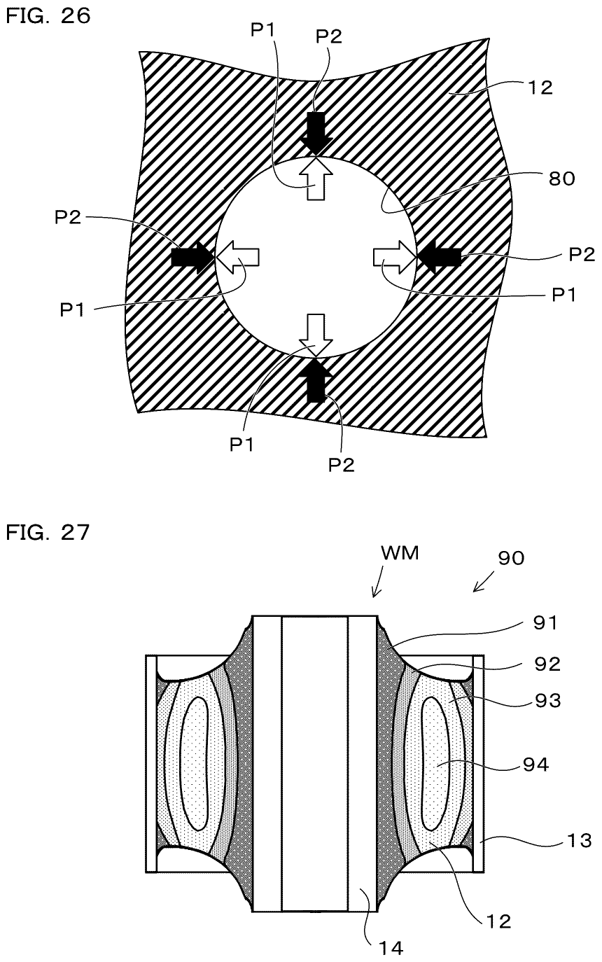

As illustrated in FIG. 26, it is considered that when pressure P1 at which the internal bubbles 80 are about to expand and pressure P2 at which the polymer portion is about to suppress the internal bubbles 80 satisfy P1<P2, the internal bubbles 80 are suppressed by rubber and disappear. Therefore, the blow point reaction rate was converted into the torque measured by the vulcanization tester 70 corresponding to the blow point reaction rate, and compared in each sample.

FIG. 25(b) shows blow point torque when a plurality of types of fillers are added to a plurality of types of polymers in different formulation amounts. As described above, the blow point torque is obtained by converting the blow point reaction rate into the torque measured by the vulcanization tester 70 corresponding to the blow point reaction rate.

As shown in FIG. 25(b), regardless of the type of the raw material polymer and the formulation amount of the filler, the torque (blow point torque) at which the generation of the internal bubbles 80 was not observed was substantially the same value. That is, use of easily measurable torque enables the blow point to be easily predicted. The threshold is described on the vertical axis in FIG. 25(b). The estimation unit 62 of the internal bubble estimation unit 6 estimates that the internal bubbles 80 are generated in the polymer portion 12 of the target work model WM when the torque at the time when the target work model WM is demolded does not exceed a threshold.

The threshold is set to a uniform value for plural types of raw material polymers. The threshold is torque corresponding to a reaction rate larger than the reaction rate of the crosslinking reaction at the blow point of the polymer portion 12. However, a plurality of groups may be set as the threshold, and the threshold may be set for each group. The plurality of groups can be divided according to any criteria.

The reaction rate acquisition unit 51 configures to acquire the reaction rate for each site of the polymer portion 12, the torque calculation unit 61 configures to calculate the torque for each site of the polymer portion 12, and the estimation unit 62 configures to estimate the generation of the internal bubbles 80 for each site of the polymer portion 12.

### 7. Display Processing S5

Next, the display processing S5 will be described. As illustrated in FIG. 1, the display unit 7 configures to display a value obtained from the reaction rate corresponding to the reaction time based on the analysis result of the crosslinking reaction analysis unit 4.

Furthermore, based on the analysis result of the structural analysis unit 5, the display unit 7 configures to display characteristics corresponding to the temperature of the forming mold model MM used for the crosslinking reaction of the polymer portion 12 and an in-mold reaction time from the start of the crosslinking reaction to the demolding of the forming mold model MM.

Furthermore, based on the estimation result of the internal bubble estimation unit 6, the display unit 7 configures to display the presence or absence of generation of the internal bubbles 80 according to the temperature of the forming mold model MM and the in-mold reaction time from start of the crosslinking reaction to demolding of the forming mold model MM.

Furthermore, based on the analysis result of the crosslinking reaction analysis unit 4, the display unit 7 configures to display, in accordance with the presence or absence of generation of the internal bubbles 80, a value obtained from the reaction rate corresponding to the temperature of the forming mold model MM and the in-mold reaction time from the start of the crosslinking reaction to the demolding of the forming mold model MM. However, the content displayed by the display unit 7 is not limited to the above.

### 7-1. Display Method by Table

### (1) Mode for Displaying Reaction Rate

With reference to Tables 1 to 4, a form in which the reaction rate of the crosslinking reaction is displayed using tables will be described.

TABLE 1

| REACTION | | REACTION TIME [MINUTES] | | | | | | | | |
|---|---|---|---|---|---|---|---|---|---|---|
| RATE [%] | | 3 | 3.5 | 4 | 4.5 | 5 | 5.5 | 6 | 6.5 | 7 |
| MOLD | 178 | 110 | 114 | 117 | 121 | 123 | 126 | 128 | 129 | 131 |
| TEMPER- | 176 | 108 | 111 | 115 | 118 | 120 | 122 | 124 | 126 | 128 |
| ATURE | 174 | 106 | 109 | 112 | 115 | 117 | 119 | 121 | 123 | 125 |
| [° C.] | 172 | 104 | 107 | 110 | 112 | 114 | 117 | 118 | 120 | 122 |
| | 170 | 102 | 105 | 107 | 110 | 112 | 114 | 116 | 117 | 119 |
| | 168 | 101 | 103 | 105 | 107 | 110 | 111 | 113 | 115 | 116 |
| | 166 | 100 | 101 | 103 | 106 | 107 | 109 | 111 | 112 | 114 |
| | 164 | 100 | 100 | 102 | 104 | 105 | 107 | 109 | 110 | 111 |
| | 162 | 99 | 100 | 100 | 102 | 104 | 105 | 106 | 108 | 109 |
| | 160 | 96 | 99 | 100 | 100 | 102 | 103 | 105 | 106 | 107 |
| | 158 | 88 | 97 | 100 | 100 | 100 | 101 | 103 | 104 | 105 |
| | 156 | 75 | 91 | 97 | 100 | 100 | 100 | 101 | 102 | 103 |
| | 154 | 47 | 79 | 92 | 97 | 100 | 100 | 100 | 101 | 102 |
| | 152 | 9 | 56 | 80 | 91 | 97 | 99 | 100 | 100 | 100 |
| | 150 | 3 | 12 | 58 | 79 | 90 | 96 | 99 | 100 | 100 |

Table 1 is a table summarizing the relation between the mold temperature and the reaction time for the reaction rate of the crosslinking reaction in the element having the minimum reaction rate of the crosslinking reaction in the target work model WM.

The reaction time described in Table 1 represents the reaction time in a state where the target work model WM is arranged inside the mold 10. The numerical values of the reaction rate described in Table 1 are calculated based on both temperature histories of the reaction time in the state where the target work model WM is arranged inside the mold 10 and the time after the target work model WM is demolded from the mold 10. Since the crosslinking reaction progresses also after the target work model WM is demolded from the mold 10, the above consideration is required. In the following description, the same applies to the reaction times and reaction rates shown in Tables 2 to 4.

In Table 1, a region surrounded by a thick ruled line in the upper right part has a reaction rate more than 115%. In this region, the return reaction of the crosslinking reaction excessively progresses, which is not preferable. However, the range of the reaction rate can be arbitrarily set, and a value different from 115% may be used as the threshold.

A region surrounded by a thick ruled line in the lower left part of Table 1 has a value of the reaction rate smaller than 95%. In this region, the crosslinking reaction does not sufficiently progress, which is not preferable. However, the range of the reaction rate can be arbitrarily set, and a value different from 95% may be used as the threshold.

In Table 1, the region except for the region of the upper right region and the lower left region, which are surrounded by the thick ruled lines, is a preferable region from the viewpoint of the reaction rate of the crosslinking reaction. Note that Table 1 does not describe information regarding the internal bubbles 80 described later.

Table 2 is a table summarizing the relation between the mold temperature and the reaction time for the mean value of the crosslinking reactions of all elements of the target work model WM.

TABLE 2

| REACTION | | REACTION TIME [MINUTES] | | | | | | | | |
|---|---|---|---|---|---|---|---|---|---|---|
| RATE [%] | | 3 | 3.5 | 4 | 4.5 | 5 | 5.5 | 6 | 6.5 | 7 |
| MOLD | 178 | 114 | 117 | 120 | 123 | 125 | 127 | 129 | 131 | 133 |
| TEMPER- | 176 | 111 | 114 | 117 | 120 | 122 | 124 | 126 | 128 | 129 |
| ATURE | 174 | 109 | 112 | 115 | 117 | 119 | 121 | 123 | 125 | 126 |
| [° C.] | 172 | 107 | 109 | 112 | 114 | 116 | 118 | 120 | 122 | 123 |
| | 170 | 105 | 107 | 110 | 112 | 114 | 115 | 117 | 119 | 120 |
| | 168 | 103 | 105 | 107 | 109 | 111 | 113 | 114 | 116 | 117 |
| | 166 | 102 | 104 | 106 | 107 | 109 | 111 | 112 | 113 | 115 |
| | 164 | 101 | 102 | 104 | 105 | 107 | 108 | 110 | 111 | 112 |
| | 162 | 100 | 101 | 102 | 104 | 105 | 106 | 108 | 109 | 110 |
| | 160 | 99 | 100 | 101 | 102 | 103 | 105 | 106 | 107 | 108 |
| | 158 | 97 | 99 | 100 | 101 | 102 | 103 | 104 | 105 | 106 |
| | 156 | 92 | 98 | 99 | 100 | 100 | 101 | 102 | 103 | 104 |
| | 154 | 83 | 93 | 98 | 99 | 100 | 100 | 101 | 102 | 103 |
| | 152 | 63 | 84 | 93 | 97 | 99 | 100 | 100 | 100 | 101 |
| | 150 | 36 | 66 | 84 | 92 | 97 | 99 | 100 | 100 | 100 |

In Table 2, a region surrounded by a thick ruled line in the upper right part has a reaction rate more than 115%. In this region, the return reaction of the crosslinking reaction excessively progresses, which is not preferable. However, the range of the reaction rate can be arbitrarily set, and a value different from 115% may be used as the threshold.

A region surrounded by a thick ruled line in the lower left part of Table 2 has a value of the reaction rate smaller than 95%. In this region, the crosslinking reaction does not sufficiently progress, which is not preferable. However, the range of the reaction rate can be arbitrarily set, and a value different from 95% may be used as the threshold.

In Table 2, the region except for the region of the upper right region and the lower left region is a preferable region from the viewpoint of the reaction rate of the crosslinking reaction. Note that Table 2 does not describe information regarding the internal bubbles 80 described later.

Table 3 is a table summarizing the relation between the mold temperature and the reaction time for the reaction rate of the crosslinking reaction in the element having the maximum reaction rate of the crosslinking reaction in the target work model WM.

TABLE 3

| REACTION | | REACTION TIME [MINUTES] | | | | | | | | |
|---|---|---|---|---|---|---|---|---|---|---|
| RATE [%] | | 3 | 3.5 | 4 | 4.5 | 5 | 5.5 | 6 | 6.5 | 7 |
| MOLD | 178 | 120 | 123 | 125 | 127 | 129 | 131 | 133 | 134 | 135 |
| TEMPER- | 176 | 117 | 120 | 122 | 124 | 126 | 128 | 129 | 131 | 132 |
| ATURE | 174 | 114 | 117 | 119 | 121 | 123 | 125 | 126 | 127 | 129 |
| [° C.] | 172 | 112 | 114 | 116 | 118 | 120 | 122 | 123 | 124 | 126 |
| | 170 | 110 | 112 | 114 | 116 | 117 | 119 | 120 | 121 | 123 |
| | 168 | 107 | 109 | 111 | 113 | 115 | 116 | 117 | 119 | 120 |
| | 166 | 106 | 107 | 109 | 111 | 112 | 113 | 115 | 116 | 117 |
| | 164 | 104 | 106 | 107 | 108 | 110 | 111 | 112 | 113 | 115 |
| | 162 | 102 | 104 | 105 | 106 | 108 | 109 | 110 | 111 | 112 |
| | 160 | 101 | 102 | 103 | 105 | 106 | 107 | 108 | 109 | 110 |
| | 158 | 100 | 101 | 102 | 103 | 104 | 105 | 106 | 107 | 108 |
| | 156 | 100 | 100 | 101 | 101 | 102 | 103 | 105 | 105 | 106 |
| | 154 | 99 | 100 | 100 | 100 | 101 | 102 | 103 | 104 | 105 |
| | 152 | 96 | 99 | 100 | 100 | 100 | 101 | 101 | 102 | 103 |
| | 150 | 88 | 95 | 98 | 100 | 100 | 100 | 100 | 101 | 101 |

In Table 3, a region surrounded by a thick ruled line in the upper right part has a reaction rate more than 115%. In this region, the return reaction of the crosslinking reaction excessively progresses, which is not preferable. However, the range of the reaction rate can be arbitrarily set, and a value different from 115% may be used as the threshold.

A region surrounded by a thick ruled line in the lower left part of Table 3 has a value of the reaction rate smaller than 95%. In this region, the crosslinking reaction does not sufficiently progress, which is not preferable. However, the range of the reaction rate can be arbitrarily set, and a value different from 95% may be used as the threshold.

In Table 3, the region except for the region of the upper right region and the lower left region is a preferable region from the viewpoint of the reaction rate of the crosslinking reaction. Note that Table 3 does not describe information regarding the internal bubbles 80 described later.

In Table 4, information regarding the internal bubbles 80 is added to Table 2.

TABLE 4

| REACTION | | REACTION TIME [MINUTES] | | | | | | | | |
|---|---|---|---|---|---|---|---|---|---|---|
| RATE [%] | | 3 | 3.5 | 4 | 4.5 | 5 | 5.5 | 6 | 6.5 | 7 |
| MOLD | 178 | 114 | 117 | 120 | 123 | 125 | 127 | 129 | 131 | 133 |
| TEMPER- | 176 | 111 | 114 | 117 | 120 | 122 | 124 | 126 | 128 | 129 |
| ATURE | 174 | 109 | 112 | 115 | 117 | 119 | 121 | 123 | 125 | 126 |
| [° C.] | 172 | 107 | 109 | 112 | 114 | 116 | 118 | 120 | 122 | 123 |
| | 170 | 105 | 107 | 110 | 112 | 114 | 115 | 117 | 119 | 120 |
| | 168 | 103 | 105 | 107 | 109 | 111 | 113 | 114 | 116 | 117 |
| | 166 | 102 | 104 | 106 | 107 | 109 | 111 | 112 | 113 | 115 |
| | 164 | 101 | 102 | 104 | 105 | 107 | 108 | 110 | 111 | 112 |
| | 162 | 100 | 101 | 102 | 104 | 105 | 106 | 108 | 109 | 110 |
| | 160 | 99 | 100 | 101 | 102 | 103 | 105 | 106 | 107 | 108 |
| | 158 | 97 | 99 | 100 | 101 | 102 | 103 | 104 | 105 | 106 |
| | 156 | 92 | 98 | 99 | 100 | 100 | 101 | 102 | 103 | 104 |
| | 154 | 83 | 93 | 98 | 99 | 100 | 100 | 101 | 102 | 103 |
| | 152 | 63 | 84 | 93 | 97 | 99 | 100 | 100 | 100 | 101 |
| | 150 | 36 | 66 | 84 | 92 | 97 | 99 | 100 | 100 | 100 |

In Table 4, a region surrounded by a thick ruled line in the upper right part has a reaction rate more than 115%. In this region, the return reaction of the crosslinking reaction excessively progresses, which is not preferable. However, the range of the reaction rate can be arbitrarily set, and a value different from 115% may be used as the threshold.

In Table 4, a part surrounded by a double ruled line indicates that the internal bubbles 80 are generated in the polymer portion 12 of the target work model WM.

In the part surrounded by a double ruled line in the lower left part of Table 4, a region below the broken line has a value of the reaction rate smaller than 95%. In this region, the crosslinking reaction does not sufficiently progress, which is not preferable. However, the range of the reaction rate can be arbitrarily set, and a value different from 95% may be used as the threshold.

In Table 4, the region above the broken line in the region surrounded by the double ruled line indicates that the reaction rate of the crosslinking reaction was in a range of 95% to 115% but the internal bubbles 80 were generated in the polymer portion 12 of the target work model WM. This region is not preferable as a product because the internal bubbles 80 were generated in the polymer portion 12.

In the present embodiment, Table 4 was created by adding the information regarding the internal bubbles 80 to the prediction result described in Table 2, but the present disclosure is not limited to this, and the information regarding the internal bubbles 80 can be added to the prediction result of the reaction rate at any temperature.

### (2) Form of Displaying Elastic Modulus

Table 5 is a table summarizing the relation between the mold temperature and the reaction time for the elastic modulus of the target work model WM.

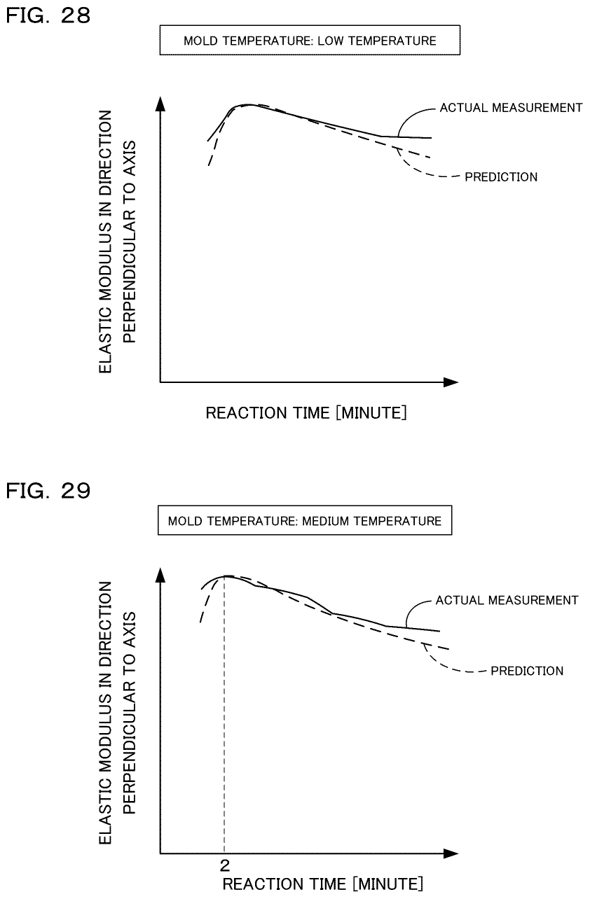

The reaction time described in Table 5 represents the reaction time in a state where the target work model WM is arranged inside the mold 10. The values of the elastic modulus described in Table 5 are calculated based on both temperature histories of the reaction time in the state where the target work model WM is arranged inside the mold 10 and the time after the target work model WM is demolded from the mold 10. Since the crosslinking reaction progresses also after the target work model WM is demolded from the mold 10, the above consideration is required. Note that in the following description, the same applies to the reaction times and the elastic moduli described in FIGS. 28 to 31.

TABLE 5

| RATIO VALUE OF ELASTIC | | | | | | | | | | |
|---|---|---|---|---|---|---|---|---|---|---|
| MODULUS IN AXIS ORTHOGONAL | | REACTION TIME [MINUTES] | | | | | | | | |
| DIRECTION | | 3 | 3.5 | 4 | 4.5 | 5 | 5.5 | 6 | 6.5 | 7 |
| MOLD | 178 | 0.82 | 0.79 | 0.76 | 0.73 | 0.71 | 0.69 | 0.67 | 0.65 | 0.64 |
| TEMPER- | 176 | 0.85 | 0.82 | 0.79 | 0.76 | 0.74 | 0.72 | 0.70 | — | 0.67 |
| ATURE | 174 | 0.88 | 0.85 | 0.82 | 0.80 | 0.78 | 0.76 | 0.74 | 0.72 | 0.71 |
| [° C.] | 172 | 0.91 | 0.88 | 0.85 | 0.83 | 0.81 | 0.79 | 0.77 | 0.76 | 0.74 |
| | 170 | 0.93 | 0.91 | 0.88 | 0.86 | 0.84 | 0.82 | 0.81 | 0.79 | 0.78 |
| | 168 | 0.95 | 0.93 | 0.91 | 0.89 | 0.87 | 0.85 | 0.84 | 0.82 | 0.81 |
| | 166 | 0.97 | 0.95 | 0.93 | 0.92 | 0.90 | 0.88 | 0.87 | 0.85 | 0.84 |
| | 164 | 0.99 | 0.98 | 0.96 | 0.94 | 0.92 | 0.91 | 0.89 | 0.88 | 0.87 |
| | 162 | 1.00 | 0.99 | 0.98 | 0.96 | 0.95 | 0.93 | 0.92 | 0.91 | 0.90 |
| | 160 | 0.99 | 1.00 | 0.99 | 0.98 | 0.97 | 0.95 | 0.94 | 0.93 | 0.92 |
| | 158 | 0.97 | 1.00 | 1.00 | 0.99 | 0.98 | 0.97 | 0.96 | 0.95 | 0.94 |
| | 156 | — | 0.98 | 1.00 | 1.00 | 1.00 | 0.99 | 0.98 | 0.97 | 0.95 |
| | 154 | 0.82 | 0.93 | 0.98 | 1.00 | 1.00 | 1.00 | 0.99 | 0.98 | 0.97 |
| | 152 | 0.69 | 0.84 | 0.93 | 0.98 | 0.99 | 1.00 | 1.00 | 1.00 | 0.99 |
| | 150 | — | 0.70 | 0.84 | 0.93 | 0.97 | 0.99 | 1.00 | 1.00 | 1.00 |

The elastic modulus is an elastic modulus when a static force is applied in a direction perpendicular to the axis A of the target work model WM. The numerical values in the table are values of the ratio to the elastic modulus when the reaction rate of the crosslinking reaction is 100%. However, the direction of the force applied to the target work model WM is not limited, and it is possible to adopt an elastic modulus in a case of applying a force in any direction, such as a case of applying a static force in a direction parallel to the axis A of the target work model WM, a case of applying a static force in a direction rotating about the axis A of the target work model WM, and a case of applying a static force in a direction inclined with respect to the axis A of the target work model WM. The force applied to the target work model WM is not limited to the static force, and may be, for example, an elastic modulus when vibration is applied to the target work model WM.

Table 5 is a table summarizing the relation between the mold temperature and the reaction time for the elastic modulus obtained as a result of performing structural analysis with the elastic modulus corresponding to the vulcanization degree being assigned to each element of the target work model WM. The meaning of the thick ruled line, the double line ruled line, and the broken ruled line described in Table 5 are the same as the content described in Table 4, and thus redundant description will be omitted. Note that in Table 5, the cell filled with “-” is a place where a calculation error occurred in the simulation of the elastic modulus.

Furthermore, any information such as information regarding the internal bubbles 80, for example, can be added to Table 5.

In the present embodiment, the ruled line is used to divide the numerical values described in the tables, but the present disclosure is not limited to this, and the numerical values in the tables can be divided by any method such as changing the color of the numerical values described in the tables, changing the fonts, changing the typeface to oblique fonts or bold fonts, changing the background color of the cells in which the numerical values are described, or changing the background pattern of the cells.

### 7-2. Display Method by Heat Map Diagram 90

With reference to FIG. 27, a form in which the reaction rate of the crosslinking reaction is displayed using a heat map diagram 90 will be described. FIG. 27 illustrates the heat map diagram 90 in which the distribution of the reaction rate of the crosslinking reaction is displayed in shading for the target work model WM.

In the heat map diagram 90, the outer joint member 13 and the inner joint member 14 of the target work model WM are displayed in white. In the target work model WM, the polymer portion 12 is divided into regions 91 to 94 according to the magnitude of the reaction rate of the crosslinking reaction and displayed, and each of the divided regions 91 to 94 is shaded and displayed according to the magnitude of the crosslinking reaction rate.

The region 91 has the highest reaction rate of the crosslinking reaction and is displayed in the darkest color tone. The region 92 has the second highest reaction rate of the crosslinking reaction and is displayed in the second darkest color tone. The region 93 has the third highest reaction rate of the crosslinking reaction and is displayed in the third darkest color tone. The region 94 has the lowest reaction rate of the crosslinking reaction and is displayed in the lightest color tone.

The display unit 7 displays the heat map diagram 90 to a worker. Regarding the reaction rate of the crosslinking reaction in the polymer portion 12 of the target work model WM, the worker can intuitively understand the distribution of the reaction rate of the crosslinking reaction of the polymer portion 12 by visually recognizing the regions 91 to 94 shaded and displayed.

The heat map diagram 90 can be prepared and output for all reaction conditions (e.g., temperature, time, and the like) related to the target work model WM. The heat map diagram 90 may have an aspect of being prepared and output only for predetermined reaction conditions.

The display method of the heat map diagram 90 by the display unit 7 is not particularly limited, and for example, a form may be adopted in which the display unit 7 displays Table 5 described above, and the worker selects each cell in Table 5 displayed, whereby the heat map diagram 90 under the reaction conditions corresponding to the cell is displayed. An aspect may be adopted in which the heat map diagram 90 corresponding to the input reaction conditions is displayed when the worker inputs the reaction conditions of the crosslinking reaction.

In the present embodiment, the heat map diagram 90 has a configuration in which the polymer portion 12 is divided into the regions 91 to 94 and displayed, but the present disclosure is not limited to this, and the polymer portion 12 may be divided into 2 to 3 or 5 or more regions and displayed. In the heat map diagram 90, a region having a high reaction rate may be displayed in a light color tone, and a region having a low reaction rate may be displayed in a dark color tone.

In the heat map diagram 90, a region having a high reaction rate may be displayed in a warm color such as red, and a region having a low reaction rate may be displayed in a cool color such as blue, or a region having a high reaction rate may be displayed in a cool color such as blue, and a region having a low reaction rate may be displayed in a warm color such as red.

### 7-3. Display Method by Graph

### (1) Form of Displaying Elastic Modulus

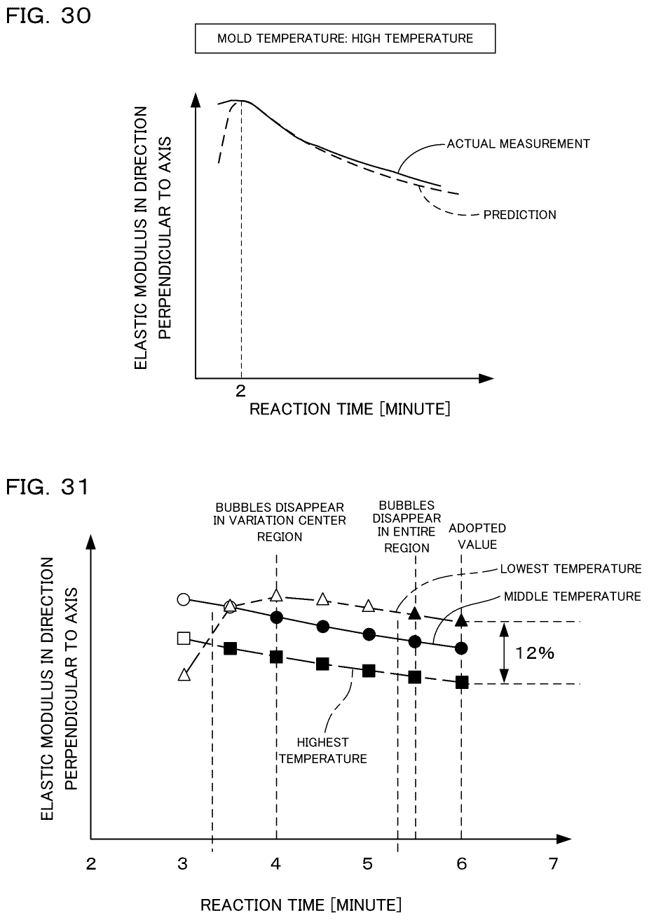

With reference to FIGS. 28 to 30, a form in which the elastic modulus of the target work model WM is displayed using a graph will be described. FIGS. 28 to 30 are graphs of the elastic modulus of the target work model WM with respect to the reaction time of the crosslinking reaction. FIG. 28 is a graph when the crosslinking reaction of the polymer portion 12 of the target work model WM is performed at the lowest temperature of the mold 10, FIG. 29 is a graph when the crosslinking reaction of the polymer portion 12 of the target work model WM is performed at a medium temperature of the mold 10, and FIG. 30 is a graph when the crosslinking reaction of the polymer portion 12 of the target work model WM is performed at the highest temperature of the mold 10. In FIGS. 28 to 30, solid lines indicate actual measurement values of the elastic modulus, and broken lines indicate prediction values.

As shown in FIGS. 28 to 30, the elastic modulus rises from the start of the crosslinking reaction to reach the maximum value, and decreases after reaching the maximum value.

In FIG. 28, in which the temperature of the mold 10 was the lowest, the actual measurement value and the prediction value of the elastic modulus accurately matched.

In FIG. 29, in which the temperature of the mold 10 was medium, the actual measurement value and the prediction value of the elastic modulus did not match until 2 minutes after the start of the reaction, but the actual measurement value and the prediction value accurately matched after 2 minutes. Until 2 minutes after the start of the reaction, the elastic modulus was predicted to be smaller than the actual measurement value. However, the region within 2 minutes after the start of the reaction is not considered to be used as a product, and thus the influence on the product is small.

In FIG. 30, in which the temperature of the mold 10 was the highest, the actual measurement value and the prediction value of the elastic modulus did not match until 2 minutes after the start of the reaction, but the actual measurement value and the prediction value accurately matched after 2 minutes. Until 2 minutes after the start of the reaction, the elastic modulus was predicted to be smaller than the actual measurement value. The difference between the actual measurement value and the prediction value was larger than that when the temperature of the mold 10 was medium. However, as described above, the region within 2 minutes after the start of the reaction is not considered to be used as a product, and thus the influence on the product is small.

### (2) Form of Displaying Elastic Modulus and Internal Bubbles 80 Together

With reference to FIG. 31, a mode in which the elastic modulus of the polymer portion 12 of the target work model WM and the prediction result regarding the generation of the internal bubbles 80 are displayed using a graph will be described. Similarly to FIG. 29 described above, the temperature of the mold 10 is set to a moderate temperature.

FIG. 31 shows a graph of the elastic modulus with respect to the reaction time when a force is applied in a direction perpendicular to the axis A of the target work model WM. The quadrangular shaped symbol indicates the elastic modulus in the element in which the temperature of the polymer portion 12 of the target work model WM has the highest value. The round shaped symbol indicates the elastic modulus in the element in which the temperature of the polymer portion 12 of the target work model WM has the median value. The triangular shaped symbol indicates the elastic modulus in the element in which the temperature of the polymer portion 12 of the target work model WM has the lowest value. In FIG. 31, the white symbol indicates a case where the internal bubbles 80 are predicted to be generated, and a black symbol indicates a case where the internal bubbles 80 are predicted not to be generated.

Note that the graph for the element in which the temperature of the polymer portion 12 has the highest value and the element in which the temperature has the median value is a graph in the reaction return period after the reaction progress period.

In the element in which the temperature of the polymer portion 12 has the highest value, the progress of the crosslinking reaction is the fastest. Therefore, it was predicted that the internal bubbles 80 were not generated when the reaction time exceeded 3 minutes and 3 minutes and 30 seconds elapsed. In the element in which the temperature of the polymer portion 12 is the highest, the progress of the return reaction of the crosslinking reaction is also fast, and thus the elastic modulus at the lapse of the reaction time of 6 minutes is the lowest.

In the element in which the temperature of the polymer portion 12 has the median value, the progress of the crosslinking reaction is also moderate. Therefore, it was predicted that the internal bubbles 80 were not generated when the reaction time exceeded 3 minutes 30 seconds and 4 minutes elapsed. The elastic modulus at the lapse of the reaction time of 6 minutes is larger than that of the element in which the temperature of the polymer portion 12 is the highest.

In the element in which the temperature of the polymer portion 12 has the lowest value, the progress of the crosslinking reaction is the slowest. Therefore, in a state where the reaction time was before 4 minutes, it was predicted to be the reaction progress period, and the elastic modulus increased with the lapse of the reaction time. After the reaction time elapsed 4 minutes, it was predicted that the return reaction of the crosslinking reaction progressed and the elastic modulus gradually decreased. In the element in which the temperature of the polymer portion 12 has the lowest value, it was predicted that the internal bubbles 80 were not generated when the reaction time exceeded 5 minutes and 5 minutes and 30 seconds elapsed. At this time point, it was predicted that the internal bubbles 80 disappeared in the entire region of the polymer portion 12 of the target work model WM. The elastic modulus at the lapse of the reaction time of 6 minutes was predicted to be the largest in the entire region of the polymer portion 12.

As described above, it was predicted that the internal bubbles 80 disappeared in the entire region of the polymer portion 12 of the target work model WM when the reaction time elapsed 5 minutes and 30 seconds, but in consideration of the risk rate, the time at the lapse of the reaction time of 6 minutes was adopted as the reaction time of this target work model WM. At the lapse of the reaction time of 6 minutes, the variation in the elastic modulus of the polymer portion 12 was predicted to be 12%.

In this manner, according to the present embodiment, for the target work model WM, the reaction time at which the internal bubbles 80 do not occur can be predicted, and variation in the elastic modulus of the polymer portion 12 at the reaction time can also be predicted.

### (3) Form of Visually Displaying Internal Bubbles 80

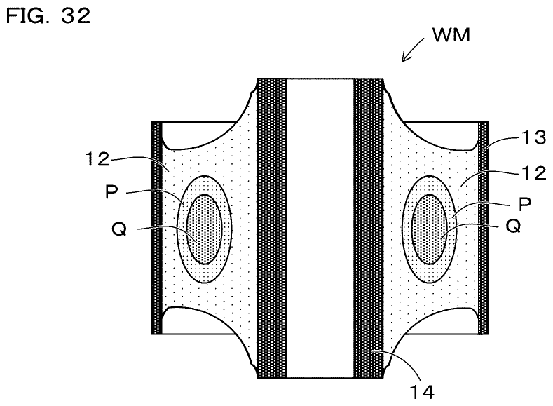

With reference to FIG. 32, a form of visually displaying the prediction result of an occurrence of the internal bubbles 80 in the polymer portion 12 of the target work model WM will be described.

FIG. 32 is a view in which the degree of possibility of generation of the internal bubbles 80 in the polymer portion 12 is displayed by shading using a cross-sectional view of the target work model WM. The outer joint member 13 and the inner joint member 14 of the target work model WM are illustrated in the darkest pattern in FIG. 32.

The polymer portion 12 of the target work model WM is illustrated in the lightest pattern in FIG. 32. A region P having an elliptical shape and a region Q having an elliptical shape positioned inside the region P are displayed inside the polymer portion 12. The region P is displayed in a pattern slightly darker than the light pattern representing the polymer portion 12. The region Q is displayed in a pattern slightly darker than the region P.

A case will be described as an example in which as a result of the internal bubble estimation processing S4, for example, the region P is determined to be a region where the internal bubbles 80 are relatively likely to be generated and the region Q is determined to be a region where the internal bubbles 80 are more likely to be generated than the region P. The regions P and Q correspond to regions where the internal bubbles 80 are actually likely to be generated (see FIG. 23).

When the internal bubble estimation processing S4 ends, the display unit 7 displays the target work model WM illustrated in FIG. 32. By viewing FIG. 32 displayed on the display unit 7, the worker visually recognizes the outer joint member 13 and the inner joint member 14 illustrated in a relatively dark pattern and the polymer portion 12 illustrated in a relatively light pattern. The worker can intuitively understand that the outer joint member 13 and the inner joint member 14 illustrated in the dark pattern are parts different from the polymer portion 12. This enables the worker to intuitively understand that it is sufficient to confirm the polymer portion 12 indicated by a relatively light pattern in order to search for the internal bubbles 80. However, the outer joint member 13 and the inner joint member 14 may be displayed in white.

Next, the worker visually recognizes that the region P and the region Q indicated by a relatively dark pattern are displayed in the polymer portion 12 indicated by a light pattern. This enables the worker to intuitively understand the internal bubbles 80 are likely to be generated in the region P and the region Q of the polymer portion 12. Furthermore, since the region Q is displayed in a darker pattern than the region P, it can be intuitively understood that the internal bubbles 80 are most likely to be generated in the region P. By this, when the manufacturing condition of the target work model WM is examined, the internal bubbles 80 may be searched in a region where the internal bubbles 80 are likely to be generated, and therefore the manufacturing condition of the target work model WM can be efficiently examined. However, a region where the internal bubbles 80 are likely to be generated may be displayed in a relatively light pattern.

The present disclosure is not limited to the above embodiment, and can be applied to various embodiments without departing from the scope of the present disclosure.

In the present embodiment, the internal bubble estimation unit 6 is configured to include the torque calculation unit 61, but is not limited to this, and may be configured to include the reaction rate acquisition unit 60 that acquires the reaction rate (vulcanization degree) of the crosslinking reaction of the polymer portion 12 of the target work model WM and the estimation unit 62 that estimates generation of a bubble in the polymer portion 12 of the target work model WM based on the reaction rate during demolding. In this case, before the reaction analysis of the vulcanization reaction is performed, the vulcanization degree at the torque serving as a threshold is obtained from the reference reaction curve RC, and this vulcanization degree can be used as a blow point vulcanization degree. The blow point vulcanization degree may be directly measured using a known measurement instrument such as a blow point analyzer or a blow point tester. Thereafter, the reaction analysis of the vulcanization reaction is performed, and the calculation result (vulcanization degree) of the reaction rate acquisition unit 60 is compared with the blow point vulcanization degree, whereby the generation of the internal bubbles can be estimated. For example, when the vulcanization degree does not exceed the blow point vulcanization degree, it is estimated that the internal bubbles 80 are generated in the polymer portion 12 of the target work model WM.

1. A crosslinking reaction simulation device comprising:
a storage unit configured to store data to be used for simulation;
a heat transfer analysis unit configured to perform heat transfer analysis of a polymer portion of a target work model during a crosslinking reaction thereof; and
a crosslinking reaction analysis unit configured to perform analysis of a reaction rate of a crosslinking reaction on the polymer portion using a result of the heat transfer analysis,
wherein the storage unit stores
the target work model including the polymer portion configured to contain a raw material polymer,
an equivalent reaction amount calculation model having a definition of an equivalent reaction amount, as a ratio of a reaction amount of a crosslinking reaction at a target reaction time at a target reaction temperature to a reaction amount of a crosslinking reaction at a reference reaction time at a reference reaction temperature, and including a slope coefficient representing a slope of an Arrhenius plot, and
the slope coefficient set corresponding to a degree of progress of a crosslinking reaction in the polymer portion, and
the crosslinking reaction analysis unit includes
a temperature acquisition unit that acquires, as a result of the heat transfer analysis, a temperature at each time for each element of the polymer portion of the target work model in a crosslinking reaction, and
a reaction rate calculation processing unit configured to calculate, on the basis of the acquired temperature of each element of the polymer portion at each time during the crosslinking reaction, the equivalent reaction amount calculation model, and the slope coefficient corresponding to the degree of progress of the crosslinking reaction at a target time, the equivalent reaction amount of the polymer portion at each time, and to calculate, on the basis of the calculated equivalent reaction amount, a reaction rate of the crosslinking reaction of the polymer portion.

2. The crosslinking reaction simulation device according to claim 1, wherein
the polymer portion is a rubber portion configured to exhibit anti-vibration performance, and
the target work model is a model of an anti-vibration rubber device.

3. The crosslinking reaction simulation device according to claim 1, wherein the slope coefficient is set to different values at a reaction progress period until a reaction rate of a crosslinking reaction reaches a peak and a reaction return period when a reaction rate exceeds a peak.

4. The crosslinking reaction simulation device according to claim 3, wherein the slope coefficient is set to a value corresponding to each division resulting from dividing an entire period including the reaction progress period and the reaction return period at equal intervals.

5. The crosslinking reaction simulation device according to claim 3, wherein in a crosslinking reaction until the reaction rate reaches a peak, the slope coefficient is set to different values respectively at a reaction progress initial period in which a crosslinking reaction starts to proceed, a reaction promotion period in which a reaction progress rate is higher than that in the reaction progress initial period, and a reaction progress later period in which the reaction progress rate is lower than that in the reaction promotion period.

6. The crosslinking reaction simulation device according to claim 3, wherein
in the slope coefficient,
the slope coefficient is set to a smaller value in the reaction return period than in the reaction progress period,
the reaction progress period is divided into a reaction progress initial period in which a crosslinking reaction starts to proceed, a reaction promotion period that is faster in reaction progress rate than the reaction progress initial period, and a reaction progress later period that is slower in reaction progress rate than the reaction promotion period, and
the slope coefficient in the reaction promotion period is set to a value larger than values of the reaction progress initial period and the reaction progress later period.

7. The crosslinking reaction simulation device according to claim 1, wherein
the slope coefficient to be used at a target time is determined based on a reaction rate at a previous time,
an increasing amount of the equivalent reaction amount during a minute time period of the target time is calculated based on a temperature at the target time, the equivalent reaction amount calculation model, and the slope coefficient, respectively at the target time,
an equivalent reaction amount integration value from start of a crosslinking reaction to the target time is calculated based on the increasing amount of the equivalent reaction amount, and
a reaction rate at the target time is calculated based on the equivalent reaction amount integration value from start of a crosslinking reaction to the target time.

8. The crosslinking reaction simulation device according to claim 7, wherein
a first relation data map defining a correspondence relation between a reaction rate of a crosslinking reaction in the polymer portion and the slope coefficient is stored, and
the slope coefficient is determined using a reaction rate at a previous time and the first relation data map.

9. The crosslinking reaction simulation device according to claim 7, wherein
a first function defining a correspondence relation between a reaction rate of a crosslinking reaction in the polymer portion and the slope coefficient is stored, and
the slope coefficient is determined using a reaction rate at a previous time and the first function.

10. The crosslinking reaction simulation device according to claim 9, wherein the first function is set to a different function corresponding to a plurality of reaction rate divisions set corresponding to a degree of progress of a crosslinking reaction in the polymer portion.

11. The crosslinking reaction simulation device according to claim 7, wherein
a second relation data map defining a correspondence relation between the equivalent reaction amount integration value and a reaction rate at a target time is stored, and
a reaction rate at a target time is determined using the equivalent reaction amount integration value at the target time and the second relation data map.

12. The crosslinking reaction simulation device according to claim 7, wherein
a second function defining a correspondence relation between the equivalent reaction amount integration value and a reaction amount at a target time is stored, and
a reaction rate at a target time is determined using the equivalent reaction amount integration value at the target time and the second function.

13. The crosslinking reaction simulation device according to claim 1, further comprising a display unit that displays a value obtained from the reaction rate corresponding to the target reaction time based on an analysis result of the crosslinking reaction analysis unit.

14. The crosslinking reaction simulation device according to claim 1, further comprising:
a structural analysis unit configured to perform structural analysis using the reaction rate of a crosslinking reaction of the polymer portion on which the analysis has been performed by the crosslinking reaction analysis unit,
wherein the structural analysis unit includes
a reaction rate acquisition unit configured to acquire the reaction rate in each element of the polymer portion calculated by the reaction rate calculation processing unit of the crosslinking reaction analysis unit,
an elastic modulus assignment unit configured to assign an elastic modulus corresponding to the reaction rate thus acquired in the polymer portion, and
a characteristic acquisition unit configured to acquire a characteristic of the target work model by performing structural analysis in a state where the elastic modulus is assigned to the polymer portion.

15. The crosslinking reaction simulation device according to claim 14, further comprising a display unit configured to display the characteristic corresponding to a temperature of a forming mold model for use in a crosslinking reaction of the polymer portion and an in-mold reaction time from start of a crosslinking reaction to demolding of a forming mold model, based on a result of the structural analysis.

16. The crosslinking reaction simulation device according to claim 1, wherein the storage unit further stores
a forming mold model,
the target work model including the polymer portion configured to further contain carbon black, and
a thermal diffusivity characteristic representing a relation between a mass ratio of the carbon black to the raw material polymer and a thermal diffusivity of the polymer portion, and
the heat transfer analysis unit includes
a condition input unit configured to input the mass ratio of the carbon black to the raw material polymer in the target work model and a temperature condition of the forming mold model,
a polymer thermal diffusivity determination unit configured to determine a polymer thermal diffusivity, which is a thermal diffusivity of the polymer portion of the target work model, based on the mass ratio input by the condition input unit and the thermal diffusivity characteristic stored in the storage unit, and
an analysis unit configured to perform heat transfer analysis on the target work model in a state of being arranged in the forming mold model, using the polymer thermal diffusivity determined by the polymer thermal diffusivity determination unit and the temperature condition stored in the storage unit.

17. The crosslinking reaction simulation device according to claim 1, wherein
the storage unit configured to further store a forming mold model,
the crosslinking reaction simulation device further comprises an internal bubble estimation unit configured to be applied to a crosslinking reaction process of causing the polymer portion of the target work model to undergo a crosslinking reaction in the forming mold model and then demolding the forming mold model, and to estimate generation of a bubble in association with demolding of the forming mold model inside the polymer portion of the target work model, and
the internal bubble estimation unit includes
a reaction rate acquisition unit configured to acquire a reaction rate of a crosslinking reaction of the polymer portion of the target work model, and
an estimation unit configured to estimate generation of a bubble inside the polymer portion of the target work model based on the reaction rate during demolding.

18. The crosslinking reaction simulation device according to claim 17, wherein
the storage unit is configured to further store
a crosslinking reaction curve defining a relation between an elapsed time from start of a crosslinking reaction and torque that has a value corresponding to a degree of progress of a crosslinking reaction in the polymer portion of the target work model, the torque being measurable by a crosslinking reaction characteristic tester using a test target polymer material corresponding to the polymer portion,
the internal bubble estimation unit is configured to further include
a torque calculation unit configured to calculate the torque corresponding to the reaction rate acquired by the reaction rate acquisition unit, based on the acquired reaction rate and the crosslinking reaction curve stored in the storage unit, and
the estimation unit is configured to estimate, on the basis of the torque during demolding, generation of a bubble inside the polymer portion of the target work model.

19. The crosslinking reaction simulation device according to claim 18, wherein
the reaction rate acquisition unit is configured to acquire the reaction rate for each site of the polymer portion,
the torque calculation unit is configured to calculate the torque for each site of the polymer portion, and
the estimation unit is configured to estimate generation of a bubble for each site of the polymer portion.

20. The crosslinking reaction simulation device according to claim 18, further comprising a display unit configured to display presence or absence of generation of the bubble in accordance with a temperature of the forming mold model and an in-mold reaction time from start of a crosslinking reaction to demolding of the forming mold model, based on an estimation result by the internal bubble estimation unit.
